(19) United States
(12) Patent Application Publication
(10) Pub. No.: US 2026/0110035 A1
(43) Pub. Date: Apr. 23, 2026

(54) DNA REPAIR SIGNATURE AND PREDICTION OF RESPONSE FOLLOWING CANCER THERAPY

(71) Applicants: Agendia N.V., Amsterdam (NL); The Regents of the University of California, Oakland, CA (US)

(72) Inventors: Annuska Maria GLAS, Assendelft (NL); Lorenza MITTEMPERGHER, Abcoude (NL); Andrei BARCARU, Almere (NL); Midas Mees KUILMAN, Purmerend (NL); Laura Johanna VAN ’T VEER, San Francisco, CA (US); Denise WOLF, Berkeley, CA (US); Hoi Sze Christina YAU, Novato, CA (US); Laura Jean ESSERMAN, San Francisco, CA (US)

(21) Appl. No.: 18/867,401

(22) PCT Filed: May 19, 2023
(86) PCT No.: PCT/NL2023/050286

§ 371 Date: Nov. 19, 2024

(30) Foreign Application Priority Data
May 19, 2022 (EP) 22174443.6

(51) Int. Cl. C12Q 1/6886; C12Q 1/6806

(52) U.S. Cl. CPC C12Q 1/6886; C12Q 1/6806; C12Q 2600/106; C12Q 2600/158

(57) ABSTRACT

The invention relates to methods for typing a sample comprising breast cancer cells or comprising gene expression products from breast cancer cells, of an individual with breast cancer. The invention further relates to methods of treating an individual with breast cancer, that is typed according to the methods of the invention.

This application is the U.S. National Phase of International Patent Application Number PCT/NL2023/050286 filed 19 May 2023, which claims priority from EP 22174443.6 filed 19 May 2022, each of which is incorporated herein by reference.

### INCORPORATION OF SEQUENCE LISTING

A computer readable XML file entitled “Substitute_Sequence_Listing_294-658_PCTUS.xml,” created on Mar. 6, 2025 with a file size of 79,846 bytes contains the Sequence Listing for this application, which has been filed with this application, and is hereby incorporated by reference in its entirety.

### FIELD OF INVENTION

The invention relates to methods for typing of cancer, especially breast cancer. The invention is directed to a set of marker genes to predict response following cancer therapy.

### 1 BACKGROUND

Cancer is a leading cause of death worldwide, and was responsible for nearly 10 million deaths in 2020. The most common cancer in 2020 in terms of new cases was breast cancer with 2.26 million cases and responsible for 685,000 deaths globally that year (Ferlay et al., 2021. Int J Cancer 10.1002/ijc.33588).

Among the most advanced treatment strategies in breast cancer (BC) are based on the inhibitors of the poly(adenosine diphosphate-ribose) polymerase (PARP) family of enzymes. In some cases tumor cells are defective for certain mechanisms (pathways) of DNA repair, these are known as DNA repair deficient (DRD) [Kneubil et al., 2022. Brazilian J Med Biol Res 55: 1-9], and by using PARP inhibitors (PARPi) [Liu et al., 2021. Front Oncol 11: 1-9, 2021; Cortesi et al., 2021. Target Oncol 16: 255-282] the BC cells are depleted from the property of self-repair and consequently are killed through apoptosis or by the infiltrated immune cells. Various PARPi drugs are known to this day amongst most common are: Veliparib, Olaparib, Rucaparib, Niraparib and Talazoparib, although at the moment only Veliparib, Olaparib and Talazoparib are used in BC treatment. Another DRD family of drugs are platinum-based compounds, of which cisplatin and carboplatin are the most known. The mechanism of action of platinum based drugs can be summarized as follows: the molecule of, for example, carboplatin enters into the cell through active transportation, once in the nucleus the molecule binds to two consecutive guanine bases creating cross-linkage (damaging DNA). Further, the molecule of carboplatin that binds to the DNA, tightly packs the HMG domain protein inserting a benzene ring between the DNA and the protein, causing further DNA damage and impairing the DNA repair mechanism [Hyochol Ahn et al., 2017. Physiol Behav 176: 139-148; Tian et al., 2021. Front Pharmacol 12: 1-13]. However, if there is an alternative mechanism of DNA repair, the tumor cell can survive. For effective treatment with a platinum-based drug, it is of high importance to detect tumors that display DNA repair deficiency (DRD+) [Wolf et al., 2017. NPI Breast Cancer 3: 1-8].

There is thus a need for a sensitive and accurate method to predict a response following PARPi, either alone or in combination with a platinum based drug, in breast cancer patients. Furthermore, such method preferably can be used on FF conserved samples, as well as on FFPE conserved.

### 2 SUMMARY

Within the ISPY2 trial [Wang and Yee, 2020. Current Breast Cancer Reports 11: 303-310], 72 patients were enrolled for treatment with Paclitaxel+Veliparib (i.e. ABT 888)+Carboplatin. Nearly 38% (n=27) of the patients showed pathological complete response (pCR). The aim of this investigation was to identify a DRD profile (here referred as DRD+) of the tumor. The expressions from microarray hybridization data of fresh frozen (FF) samples were available for the development. The resulting signature, called RePrint, proved high accuracy in leave-one-out cross-validation (LOOCV) method and high stability on the MammaPrint control samples. Pathway and gene ontology analysis using DAVID indicate the biological relevance of the selected 60 genes [Sherman et al., 2022. Nucleic Acids Research: 10.1093/nar/gkac194].

The invention provides a method of typing a sample of an individual with breast cancer, said sample comprising breast cancer cells or comprising gene expression products from breast cancer cells, the method comprising (i) isolating RNA from the sample obtained from the individual; (ii) determining an expression level of at least 7 marker genes to thereby provide an expression profile of the marker genes, wherein the marker genes are selected from the genes listed in Table 1: (iii) comparing the individual's expression profile to a reference expression profile of the at least 7 marker genes; thereby typing the sample for a response to subsequent treatment with a poly ADP ribose polymerase (PARP) inhibitor.

Said sample preferably is a fresh frozen or a formalin-fixed paraffin-embedded sample.

An individual who is typed as positively responding to a PARP inhibitor may be treated with a PARP inhibitor. Said PARP inhibitor may be combined with a platinum-based compound for treatment of said individual.

Said expression profile of the at least 5 marker genes may be performed using RNA-sequencing or microarray gene expression analysis.

Said expression profile preferably comprises at least 5 different marker genes, at least 7 different market genes, at least 8 different market genes, at least 9 different market genes, at least 10 different marker genes, at least 11 different market genes, at least 20 different marker genes, at least 30 different marker genes, at least 40 different marker genes, at least 50 different marker genes selected from the genes listed in Table 1, or all marker genes listed in Table 1.

Said reference expression profile may be composed of the average expression levels of the at least 5 marker genes specified in step (ii) of individuals having a positive response to a PARP inhibitor; the average expression levels of the at least 5 marker genes specified in step (ii) of individuals not having a positive response to a PARP inhibitor; or the average expression levels of the at least 5 marker genes specified in step (ii) of a mixture of individuals having a positive response to a PARP inhibitor and individuals not having a positive response to a PARP inhibitor.

The expression profile of an individual may be compared to two reference expression profiles, wherein one reference expression profile is composed of the average expression levels of the marker genes specified in step (ii) of individuals having a positive response to a PARP inhibitor and the other reference expression profile is composed of the average expression level of the marker genes specified in step (ii) of individuals not having a positive response to a PARP inhibitor.

A positive response following treatment with a PARP inhibitor may be a pathologic complete response (pCR).

The invention further provides a method of treating an individual with breast cancer, comprising typing of a sample from said individual using a method of the invention described herein above; treating the individual that is typed as having a positive response following treatment with a PARP inhibitor with a PARP inhibitor, optionally in combination with a platinum-based compound and/or a taxane; and treating the individual that is typed as not having a positive response following treatment with a PARP inhibitor with chemotherapy, immunotherapy, or a combination thereof.

Said PARP inhibitor preferably is or may comprise veliparib.

Said PARP inhibitor preferably is or may comprise olaparib.

Said PARP inhibitor preferably is or may comprise talazoparib.

In methods of treatment according to the invention, the PARP inhibitor may be combined with a platinum-based compound and/or a taxane. Said platinum-based compound preferably is or comprises carboplatin. Said taxane preferably is or comprises paclitaxel.

### 3 BRIEF DESCRIPTION OF THE DRAWINGS

FIG. 1. Workflow used for RePrint development. (FIG. 1A) The data is composed of pCR (squares) and RD samples (circles) that are further balanced by HR status (light grey and dark grey, respectively, in the illustration). Shown is the experimental design for one gene in one leave-one-out-cross-validation (LOOCV) iteration. (FIG. 1B) The workflow consist of balancing by HR status and template calculation with one sample left out at each iteration (step a), filtering the genes in the template by average effect size (ES) (step b) and prediction with the final 60 genes (step c).

FIG. 2. Significantly activated pathways in the comparison between “DRD+RD” vs “DRD+pCR”.

### 4 DETAILED DESCRIPTION OF THE INVENTION

### 4.1 Definitions

As is used herein, the term “cancer”, refers to a disease or disorder resulting from the proliferation of oncogenically transformed cells.

As is used herein, the term “breast cancer”, refers to a cancer originating from cells of the breasts.

As is used herein, the term “sample”, refers to any sample that can be completely or partly obtained from an individual by various means including, for example, biopsy such as needle biopsy and surgery. The term comprises any sample comprising breast cancer cells from an individual, or suspected to comprise breast cancer cells from an individual, such as a tumour or liquid biopsy. Preferably, at least 5% of the sample consists of breast cancer cells. More preferably at least 10%, 20% or 30% of the sample consists of breast cancer cells. The term “sample” further comprises any sample that may comprise gene expression products from breast cancer cells from an individual, such as blood and educated thrombocytes and/or erythrocytes.

As is used herein, the term “fresh frozen”, refers to a sample that was frozen after collection, preferably immediately frozen after collection, and conserved in frozen state thereafter.

As is used herein, the term “formalin-fixed paraffin-embedded”, refers to a sample that is processed by fixation in formalin and embedding in paraffin upon collection.

As is used herein, the term “typing of a sample”, refers to the classification of a sample based on characterized features. In this invention typing includes the characterisation of expression levels of genes in a sample assisting in the prediction of a response following treatment with a poly ADP ribose polymerase (PARP) inhibitor.

As is used herein, the term “individual”, refers to a human. Said individual preferably is a woman.

As is used herein, the term “adjuvant therapy”, refers to treatment given following a primary treatment such as surgery. An aim of adjuvant therapy is, for example, to remove cancer cells that remained after primary treatment and/or to reduce the chance of recurrence of cancer cells. Adjuvant therapy in breast cancer, in addition to surgery, involves treatment including one or more of chemotherapy, radiotherapy, immune therapy, targeted therapy and hormone therapy.

As is used herein, the term “neoadjuvant therapy”, refers to treatment that is administered prior to primary treatment such as surgery. The main aim of neoadjuvant therapy in breast cancer is to render the primary treatment easier or more effective, for example by reducing the tumour size before surgery. Neoadjuvant therapy in breast cancer involves treatment including one or more of chemotherapy, radiotherapy, immune therapy, targeted therapy and hormone therapy.

As is used herein, the term “immune therapy”, or immunotherapy, refers to treatment with one or more immunotherapeutic agents that activate or suppress the immune system. Immune therapy includes a wide range of treatments such as immune check point inhibitors, vaccines, cytokines and monoclonal antibodies.

As is used herein, the term “immune checkpoint inhibitor”, refers to an inhibitor of an immune checkpoint molecule, a regulator of the immune system. Immune checkpoint molecules include CTLA4, PD-1 and PD-L1, A2AR, CD276, B7-H4, CD272 and Herpesvirus Entry Mediator (HVEM), LAG3, NOX2, TIM-3, V-domain Ig suppressor of T cell activation (VISTA), and CD328. A preferred immune checkpoint inhibitor is selective for at least one of CTLA4, PD-1 and PD-L1, A2AR, CD276, B7-H4, CD272 and Herpesvirus Entry Mediator (HVEM), LAG3, NOX2, TIM-3, V-domain Ig suppressor of T cell activation (VISTA), and CD328, when compared to other surface molecules, meaning that the inhibitor is at least two times more potent, preferably at least five times more potent, in inhibiting at least one of CTLA4, PD-1 and PD-L1, A2AR, CD276, B7-H4, CD272 and Herpesvirus Entry Mediator (HVEM), LAG3, NOX2, TIM-3, V-domain Ig suppressor of T cell activation (VISTA), and CD328, when compared to other molecules.

As is used herein, the term “Poly [ADP-ribose]polymerase (PARP) inhibitor”, or “PARPi”, refers to an inhibitor of a poly [ADP-ribose]polymerase. PARP is a key factor in the initiation of a repair response to single-strand DNA breaks (SSB). A preferred PARP inhibitor is selective for PARP1 and/or PARP2, when compared to other polymerases, meaning that the inhibitor is at least two times more potent, preferably at least five times more potent, in inhibiting PARPi and/or PARP2, when compared to other polymerases.

As is used herein, the term “chemotherapy”, as used herein, refers to treatment with one or more chemotherapeutic agents such as alkylating agents, anthracyclines, taxanes, histone deacetylase inhibitors, topoisomerase inhibitors and platinum-based agents. Traditional chemotherapeutic agents, as used in cancer treatment, are cytotoxic and primarily kill cancer cells by inhibiting cell division.

As is used herein, the term “pathologic complete response (pCR)”, refers to the absence of any sign of cancer in an individual with breast cancer. The term pCR may be defined as the absence of residual invasive and in situ cancer, for example as determined by hematoxylin and eosin evaluation of one or more biopsies and/or regional lymph nodes following completion of therapy.

As is used herein, the term “biopsy” refers to a biopsy derived from a primary breast cancer, while the term “liquid biopsy” refers to a biopsy obtained from a bodily fluid comprising circulating breast cancer cells or cells that have absorbed nucleic acids derived therefrom such as educated thrombocytes and/or erythrocytes (Best et al., 2015. Cancer Cell 28: 666-676; Heinhuis et al., 2020. Cancers 12: 1372).

As is used herein, the term “RNA”, refers to ribonucleic acid.

As is used herein, the term “isolating RNA”, refers to the extraction and purification of RNA from a biological sample. The term “isolating” refers to the removal of other components, such as proteins and DNA, at least to some extent. As is used herein, the term “gene expression level”, refers to a quantifiable level of expression of a gene of interest A gene's expression level is often inferred by measuring a level of a gene product, such as mRNA or protein, of that gene in a sample. Said gene expression level can be determined relatively, in relation to the expression levels of other genes, such as household genes or normalization genes as described in, for example, international patent application WO2008039071; or absolutely, for example by comparing a determined level of expression to a calibration curve of the expression product of the gene.

As is used herein, the term “expression profile”, refers to the expression levels of multiple genes in a sample. An expression profile can be obtained, for example, by analysing the hybridisation pattern of a sample on a microarray, or by techniques such as RNA-sequencing and multiplex qPCR.

As is used herein, the term “marker gene”, refers to a gene whose sequence or expression level, alone or in combination with other genes, is correlated with an effect, in this application a probability of a positive or negative response following treatment with a poly ADP ribose polymerase (PARP) inhibitor.

As is used herein, the term “oestrogen-receptor (ER) positive breast cancer”, refers to a breast cancer that detectably expresses oestrogen receptor (ER). ER status may be determined, for example, by IHC and/or by TargetPrint® analysis as previously reported (Roepman et al., 2009. Clin Cancer Res 15: 7004-7011).

As is used herein, the term “oestrogen-receptor (ER) negative breast cancer”, refers to a breast cancer that does not detectably expresses oestrogen receptor (ER). ER status may be determined, for example, by IHC and/or by TargetPrint® analysis as previously reported (Roepman et al, 2009. Clin Cancer Res 15: 7004-7011).

As is used herein, the term “human epidermal growth factor receptor 2 (HER2) negative breast cancer”, refers to a breast cancer that does not detectably express human epidermal growth factor receptor 2 (HER2). HER2 is also termed v-erb-b2 avian erythroblastic leukaemia viral oncogene homolog 2 (ERBB2) or NEU. HER2 status may be determined, for example, by immunohistochemistry, chromogenic or fluorescence in situ hybridization, and/or by TargetPrint® analysis as previously reported (Roepman et al., 2009. Clin Cancer Res 15: 7004-7011).

As is used herein, the term “microarray gene expression analysis”, refers to the analysis of gene expression levels of a predefined gene set through hybridization. Microarrays, also known as chips, are microscopic slides containing microscopic spots of nucleic acid molecules from a specific gene. The nucleic acid molecules attached to the microarray act as probes for a nucleic acid molecule such as RNA or copy-DNA (cDNA) molecule, from an experimental sample. These cDNA molecules may be labelled, for example fluorescently labelled, prior to hybridization to the microarray.

The term “hybridization”, as is used herein, refers to the binding of a nucleic acid molecule such as RNA or cDNA molecule to a (partially) complementary nucleic acid probe on the microarray. Hybridization of a labelled nucleic acid molecule may result in a signal, for example a fluorescent signal, that can be detected and quantified, yielding information about the abundance of the labelled nucleic acid molecule in the experimental sample. Microarray analysis allows for the simultaneous detection of gene expression levels of a large number of genes.

As is used herein, the term “amplification”, refers to an increase in the number of copies of a particular DNA fragment through replication using at least one primer and a DNA polymerase. Known amplification methods include polymerase chain reaction (PCR) and isothermal amplification including, for example, helicase-dependent amplification (HDA) (Vincent et al., 2004. EMBO Rep 5: 795-800), loop-mediated amplification (LAMP) (Notomi et al., 2000. Nucleic Acids Res 28: E63), nucleic acid sequences-based amplification (NASBA) (Guatelli et al., 1990. Proc Nati Acad Sci USA 87: 1874-1878), rolling circle amplification (Ali et al., 2014. Chem Soc Rev 43: 3324-3341), strand-displacement amplification (SDA) (Walker et al., 1992. Nucleic Acids Res 20: 1691-6) and recombinase polymerase amplification (RPA) (Piepenburg et al., 2006. PLoS Biology 4: e204).

As is used herein, the term “RNA-Seq”, also termed “RNA-sequencing”, refers to a sequencing technique, including a high-throughput sequencing technique, preferably using next-generation sequencing (NGS), to characterize the quantity and/or sequence of a nucleic acid molecule such as RNA in a sample. RNA-Seq can be used for gene expression analysis

As is used herein, the term “normalisation”, refers to methods for correcting experimental variation and bias. Normalisation processes are for example important for analysis of large scale expression data, as collected using microarray or RNA-seq gene expression analysis, to preserve biological variation and eliminate experimental bias or technical variation.

As is used herein, the term “combination” refers to the administration of effective amounts of compounds to a patient in need thereof. Said compounds may be provided in one pharmaceutical preparation, or as two or more distinct pharmaceutical preparations. Said compounds may be administrated simultaneously, separately, or sequentially to each other. When administered as two or more distinct pharmaceutical preparations, they may be administered on the same day or on different days to a patient in need thereof, and using a similar or dissimilar administration protocol, e.g. daily, twice daily, biweekly, orally and/or by infusion. Said combination is preferably administered repeatedly according to a protocol that depends on the patient to be treated (age, weight, treatment history, etc.), which can be determined by a skilled physician. Said protocol may include daily administration for 1-30 days, such as 2 days, 10 days, or 21 days, followed by period of 1-14 days, such as 7 days, in which no compound is administered.

As used herein, the term ‘typing a sample’ refers to predicting a response to a PARP inhibitor.

As used herein, the term ‘following treatment with’ refers to typing a sample obtained from an individual to a subsequent treatment with a PARP inhibitor.

### 4.2 Sample Collection and Pre-Processing

This invention is directed to a new signature, termed RePrint, that is able to predict response to PARPi combined to platinum based chemotherapy with an overall accuracy of 94% in the VC ISPY2 arm. The RePrint genes cover various relevant pathways and mechanism such as homologous recombination, non-homologous end-joining, mismatch excision repair, nucleotide excision repair, base excision repair, Fanconi anemia, DNA polymerases, ubiquitination and modification, and others. The reporting scale of the signature ranges between 0 and 100 with a decision threshold at 50 with RePrint. A value above 50 indicates a DRD+/pCR and equal or below 50 a DRD−/RD phenotype. The value of 50 indicates that there is sufficient biological background (favorable to DRD) in the tumor to have a pCR when a PARP inhibitor/platinum based treatment will be applied. It does not reflect a percentage of any kind and no quantitative value was yet linked to the index.

The large prevalence of the DRD+ according to the RePrint score, indicates a high clinical utility of the signature. The high accuracy on the VC dataset together with the stability of the signature (over 98% stability and approximately 3% coefficient of variation (CV)) in the reproducibility samples indicates potential usage as predictive test for a treatment with PARP inhibitors and platinum-based treatment for the early stage BC patients. It is noted that a RePrint positive score does not always guarantee perfect pCR prediction as there are various mechanisms of drug resistance [Stewart, 2007. Crit Rev Oncol Hematol 63: 12-31; Li et al., 2020. Mol Cancer 19: 1-16].

In the invention gene expression molecules such as RNA molecules can be isolated from a sample comprising breast cancer cells or comprising breast cancer derived nucleic acids of an individual with breast cancer.

The sample may be obtained from an individual with breast cancer. The individual preferably is a woman. Said individual with breast cancer can be an individual diagnosed with breast cancer or likely to be diagnosed with breast cancer. Said individual with breast cancer is an individual suffering from breast cancer or likely to suffer from breast cancer. The sample may comprise any sample comprising breast cancer cells or breast cancer derived nucleic acids from said individual such as a tumour or liquid biopsy.

Said tumour biopsy can be obtained by in numerous ways, as is known to a person skilled in the art. Preferably, the biopsy is obtained using needle biopsy or surgical biopsy. During needle biopsy, cancer cells are extracted from a breast cancer using a needle. During surgical biopsy, cells are extracted from the breast cancer after making an incision in the skin. In individuals with breast cancer, surgical biopsy is often part of the primary treatment, in which the cancer, or a part thereof, is removed from the body. It is explicitly stated that the act of removing a breast cancer, or a part of a breast cancer, from an individual is not part of this invention.

Several body fluids can potentially contain circulating breast tumour cells such as blood, plasma, serum, lymphatic fluid, saliva, faeces, urine and cerebrospinal fluid. Preferably, blood or plasma is used as bodily fluid to provide a liquid biopsy of breast cancer.

The sample may be collected in any clinically acceptable manner, but is preferably collected and conserved upon isolation such as to preserve at least RNA. RNA can be obtained from a sample immediately upon harvesting, or from a conserved sample. A sample can be conserved by fixation e.g. in formalin and/or by treating the sample with an RNase inhibitor, such as RNasin (Promega) and RNasecure (Invitrogen), or an RNA stabilisation agent, such as RNAlater (Invitrogen). Preferred conservation methods of samples include fresh frozen (FF) conservation, for example in dry ice or in liquid nitrogen, and formalin-fixed paraffin-embedded (FFPE) conservation.

RNA can be isolated from a sample by methods known in the art. There are three main categories of RNA extraction techniques known to date: organic extraction involving a chaotropic agent such as guanidinium thiocyanate or guanidinium isothiocyanate, followed by, for example, phenol-chloroform extraction, silica-based column techniques (e.g. RNeasy Kit by Qiagen) and magnetic beads-based techniques (e.g. Dynabeads by Invitrogen). A preferred method involves guanidinium thiocyanate-extraction such as, e.g. TRIzol® Kit by Invitrogen.

Several methods are known to generate total RNA that is depleted from ribosomal RNA (rRNA). Depletion of the highly abundant rRNA fraction (80-90%) is desirable prior to performing an RNA-seq reaction, so that sequencing can be directed to sequencing of messenger RNAs, also termed “transcriptome”. Conventional methods for eliminating rRNA from total RNA samples include enrichment of polyadenylated (poly(A)) transcripts, and targeted depletion of rRNA. Targeted depletion is usually achieved by either rRNA pull-out using biotinylated sequence-specific probes (e.g., Illumina's Ribo-Zero and Thermo Fisher's RiboMinus) or RNase H-mediated degradation (e.g., NEB's NEBNext).

The step of isolating RNA from a sample obtained from an individual may be replaced by the provision of an RNA sample from an individual, thereby providing a method of typing a sample comprising gene expression products from breast cancer cells of an individual who is diagnosed with breast cancer, comprising (i) determining an expression level of at least 7 marker genes in the RNA sample to thereby provide an expression profile of said marker genes, wherein the marker genes are selected from the genes listed in Table 1; and (ii) comparing the individual's expression profile to a reference expression profile of the at least 7 marker genes; thereby typing the sample for a response following auxiliary immune therapy.

### 4.3. Marker Genes

The invention provides a set of at least 7 marker genes whose expression is correlated with a response, here pCR, following treatment of a breast cancer patient with a poly ADP ribose polymerase (PARP) inhibitor. Said at least 7 marker genes are selected from the list of genes provided in Table 1. Since not all breast cancer patients may benefit from treatment of a breast cancer patient with a PARP inhibitor, predicting a response before the actual start of the treatment may be part of an approach for optimal treatment of said individual. Said prediction may help a physician in selecting a treatment strategy for said individual. Preferably, a set of at least 5, 8, 9, 10, 11, 20, 30, 40, 50 or 60 marker genes from the marker genes listed in Table 1 is used, such as all 60 genes listed in Table 1.

A preferred set of marker genes would comprise 3 marker genes with a positive correlation to RD and 4 marker genes with a positive correlation to pCR. Such combination includes MSH3, RAD17, NME3, EXO1 SMC6, FANCC, MSH6. Another preferred set of marker genes comprises 4 marker genes with a positive correlation to RD and 3 marker genes with a positive correlation to pCR. Such combination includes MSH3, RAD17, NME3, MRDH8, EXO1 SMC6, FANCC. Another preferred set of marker genes would comprise 5 marker genes with a positive correlation to RD and 2 marker genes with a positive correlation to pCR. Such combination includes MSH3, RAD17, NME3, MRDH8, ADAM32, EX01 SMC6. Another preferred set of marker genes comprises 2 marker genes with a positive correlation to RD and 5 marker genes with a positive correlation to pCR. Such combination includes MSH3, RAD17, EXO1, SMC6, FANCC, MSH6, FANCA. Another preferred set of marker genes comprises 6 marker genes with a positive correlation to RD and 1 marker gene with a positive correlation to pCR. Such combination includes MSH3, RAD17, NME3, MRDH8, ADAM32, BRCA1, EX01. Another preferred set of marker genes comprises 1 marker gene with a positive correlation to RD and 6 marker genes with a positive correlation to pCR. Such combination includes MSH3, EXO1 SMC6, FANCC, MSH6, FANCA, CD209. Another preferred set of marker genes comprises 7 marker genes with a positive correlation to RD and no marker genes with a positive correlation to pCR. Such combination includes MSH3, RAD17, NME3, MRDH8, ADAM32, BRCA1, COPS9. Another preferred set of marker genes comprises no marker genes with a positive correlation to RD and 7 marker genes with a positive correlation to pCR. Such combination includes EX01 SMC6, FANCC, MSH6, FANCA, CD209, LIG3.

The marker genes are provided in Table 1 with RefSeq Entrez ID, gene symbol, probe sequence, Ensemble ID, Human Genome Nomenclature Committee (HGNC) ID, and their correlation to pCR following treatment of a breast cancer patient with a PARP inhibitor. In case of a discrepancy in the identification of a marker gene, the sequence of the probe is dominant. A positive correlation to pCR (indicated as “up” in Table 1) means that the level of expression of a gene is increased in a patient that positively responded to treatment with a PARP inhibitor, when compared to a control. A positive correlation to RD means that the level of expression of a gene is increased in a patient that did not respond to treatment with a PARP inhibitor, when compared to a control.

TABLE 1
Overview of gene markers to predict pathologic complete response (pCR) following treatment
with a PARP inhibitor (SEQ ID Nos 1-60, respectively top to bottom, see first column).

| | | Gene | | | HGNC | Up |
|---|---|---|---|---|---|---|
| | Entrez ID | Symbol | Probe Sequence | ENSEMBL ID | ID | in |
| 1 | NM_002439 | MSH3 | AAACTAGCAGATGTTCCTGGAGAAATTTTGAAGAAAGCAGC | ENSG00000113318 | 7326 | RD |
| | | | TCACAAGTCAAAAGAGCTG | | | |
| 2 | NM_130398 | EXO1 | AAGCATCCAGAAGAGAAAGCATCATAATGCCGAGAACAAGC | ENSG00000174371 | 3511 | pCR |
| | | | CGGGGTTACAGATCAAACT | | | |
| 3 | NM_024624 | SMC6 | TCCTTTCAGATGCCTGGATGAATTTGATGTCTACATGGATAT | ENSG00000163029 | 20466 | pCR |
| | | | GGTTAATAGGAGAATTGC | | | |
| 4 | NM_000136 | FANCC | GAATCTGACCTTACTCTAGCAGGAGTTTTTGTAGACTCTCCC | ENSG00000158169 | 3584 | pCR |
| | | | TGATAGTTTAGTTTTTGA | | | |
| 5 | NM_000179 | MSH6 | GAAGTTTGCCTGGCTAGTGAAAGGTCAACTGTAGATGCTGA | ENSG00000116062 | 7329 | pCR |
| | | | AGCTGTCCATAAATTGCTG | | | |
| 6 | NM_000135 | FANCA | ATGAGGGCCTCGTTTATTAAGATCTTTAAACTGCTTTATACA | ENSG00000187741 | 3582 | pCR |
| | | | CTGTCACGTGGCTTCATC | | | |
| 7 | NM_021155 | CD209 | AGGGCGGCCCTGTACTTTTTGTGGCTTGGTTTCTCTGGAAAT | ENSG00000090659 | 1641 | pCR |
| | | | GTCACCTTTTCGGGCGCA | | | |
| 8 | NM_002311 | LIG3 | GGAAATCTGCCACTAACCTTCCCCAACTCAAGGAACTGTACC | ENSG00000005156 | 6600 | pCR |
| | | | AGTTGTCCAAGGAGAAGG | | | |
| 9 | NM_005742 | PDIA6 | CGCAAGATGAAATTTGCTCTGCTAAAAGGCTCCTTCAGTGAG | ENSG00000143870 | 30168 | pCR |
| | | | CAAGGCATCAACGAGTTT | | | |
| 10 | NM_002873 | RAD17 | GTGAGTGGTTTAAGGAGCGGTCAGTGTGTATAAAGTGTGTT | ENSG00000152942 | 9807 | RD |
| | | | TGAACATTATGCCAAATAT | | | |
| 11 | NM_080649 | APEX1 | AAGAAAGGATTAGATTGGGTAAAGGAAGAAGCCCCAGATAT | ENSG00000100823 | 587 | pCR |
| | | | ACTGTGCCTTCAAGAGACC | | | |
| 12 | NM_003368 | USP1 | TTGGGCATGGACTAATTTGTATCTGTTTAACTCATATTCTGC | ENSG00000162607 | 12607 | pCR |
| | | | ACGATCTGTATATAGTAC | | | |
| 13 | NM_002513 | NME3 | ACACTGTCCTGGTGCCCAGCCCAACGTGGTCCAACGTTTTTT | ENSG00000103024 | 7851 | RD |
| | | | TATAATAAAGTCCTCGTT | | | |
| 14 | NM_152503 | MROH8 | AGAATTCCAGTCTTGAGAAACAGGTCAAGGACAGCTTCAAA | ENSG00000101353 | 16125 | RD |
| | | | AGAGATTCTAAATAAATGT | | | |
| 15 | NM_001469 | XRCC6 | TTTATGTTTTTGAGGCTTTCTGTTGCCATGGTGATGGTGTAG | ENSG00000196419 | 4055 | pCR |
| | | | CCCTCCCACTTTGCTGTT | | | |
| 16 | NM_139178 | ALKBH3 | TTACTCTGCAATCTTTATCGCAATGAGAAGGACAGCGTGGAC | ENSG00000166199 | 30141 | pCR |
| | | | TGGCACAGTGATGATGAA | | | |
| 17 | NM_014812 | CEP170 | TGCTGCTGTTTCAGCCGCAGCTGAATTTGAGAATGCTGAATC | ENSG00000143702 | 28920 | pCR |
| | | | TGAGGCTGATTTCAGTAT | | | |
| 18 | NM_032043 | BRIP1 | ATGACCTAGCTGAAACTGATAGAGGAAATAGATTGGCTAAC | ENSG00000136492 | 20473 | pCR |
| | | | AATTCAGATTGCATTTTAG | | | |
| 19 | NM_004584 | RAD9A | CAAGTTTTCCTTGCTTTCCTGATACTCTTTGGCGCTGACTTG | ENSG00000172613 | 9827 | pCR |
| | | | GAATTCTAAGAGCCTTGG | | | |
| 20 | NM_145004 | ADAM32 | AACCAGATCAGAAAGCAGCAGTCAAGCTGATACTAGCAAATC | ENSG00000197140 | 15479 | RD |
| | | | CAAATCAGAAGATAGTGC | | | |
| 21 | NM_007295 | BRCA1 | AAGTGTTTTTCATAAACCCATTATCCAGGACTGTTTATAGCT | ENSG00000012048 | 1100 | RD |
| | | | GTTGGAAGGACTAGGTCT | | | |
| 22 | NM_145648 | SLC15A4 | AACGGCTGCTATTTGAACTATTACTTTTTTCTTCTGGCTGCT | ENSG00000139370 | 23090 | pCR |
| | | | ATTCAAGGAGCTACCCTC | | | |
| 23 | NM_003958 | RNF8 | TCTGCTCCTACTGTATCAATGAATGGATGAAGCGGAAGATAG | ENSG00000112130 | 10071 | pCR |
| | | | AATGCCCCATTTGTCGGA | | | |
| 24 | AF487338 | COPS9 | AGGACCTGTTTAACCAGAAAGATAAGACTGTTCTTAACGACC | ENSG00000172428 | 21314 | RD |
| | | | TAGATGATTCTGTTCATC | | | |
| 25 | NM_000444 | PHEX | TCATTATTTGGGTAGGTGACCTGCTTGGATCTAGACAGCATC | ENSG00000102174 | 8918 | pCR |
| | | | TGTTCAAAGTTGTAGGGC | | | |
| 26 | NM_152617 | RNF168 | CACTCCCTACAGCCTAGCATTTCACAGAAAAGTGTTTTTCAG | ENSG00000163961 | 26661 | pCR |
| | | | ATGTTTCAGAGATGCACA | | | |
| 27 | NM_002105 | H2AFX | CTAGAACCTTAGGCATTGGGGAGTTTTAGATGGACTAATTTT | ENSG00000188486 | 4739 | pCR |
| | | | ATTAAAGGATTGTTTTTT | | | |
| 28 | NM_021922 | FANCE | GGGTACCAGTGGGCTCAGATGAAGCTCTTGTGCTCATGTATA | ENSG00000112039 | 3586 | pCR |
| | | | TTATGACCTTCTGTGTTT | | | |
| 29 | NM_012302 | ADGRL2 | CCACATGCGAGTATTAATAAATAAAGACACCATTGGCCTGAC | ENSG00000117114 | 18582 | pCR |
| | | | GCAGCTCCCTCAAACTCT | | | |
| 30 | NM_004251 | RAB9A | CTTTGAGGAAGCGGTTCGAAGAGTTCTTGCTACCGAGGATAG | ENSG00000123595 | 9792 | pCR |
| | | | GTCAGATCATTTGATTCA | | | |
| 31 | NM_002592 | PCNA | ACCCCTTGTTGTAGAGTATAAAATTGCGGATATGGGACACTT | ENSG00000132646 | 8729 | pCR |
| | | | AAAATACTACTTGGCTCC | | | |
| 32 | NM_006357 | UBE2E3 | ATGAAGGTGGTGTGTTTTTTCTGGATATCACATTTTCATCAG | ENSG00000170035 | 12479 | pCR |
| | | | ATTATCCATTTAAGCCAC | | | |
| 33 | NM_012222 | MUTYH | ATGGGCCAGCAAGTCCTGGATAATTTCTTTCGGTCTCACATC | ENSG00000132781 | 7527 | pCR |
| | | | TCCACTGATGCACACAGC | | | |
| 34 | NM_000251 | MSH2 | AACCCTTTTTCCATAGTGTTAACTGTCAGTGCCCATGGGCTA | ENSG00000095002 | 7325 | pCR |
| | | | TCAACTTAATAAGATATT | | | |
| 35 | NM_001005207 | TRIM37 | TTGACTCCTTTTCAACCATTTGTCACAGACGTGAGAAGAAGA | ENSG00000108395 | 7523 | RD |
| | | | AATGACTTCAAAATCAAG | | | |
| 36 | NM_002616 | PER1 | TTCTCAGAACTCAGATGTGGCTAGACCAACCAGTGGGAAACT | ENSG00000179094 | 8845 | pCR |
| | | | GCCCCAGCTTCTCCCACC | | | |
| 37 | NM_002412 | MGMT | TCTTCACCATCCCGTTTTCCAGCAAGAGTCGTTCACCAGACA | ENSG00000170430 | 7059 | RD |
| | | | GGTGTTATGGAAGCTGCT | | | |
| 38 | BC066344 | ZSWIM7 | AGCAGCTAAGTGTCTCTGACAAGCAGTTGACTGACATATTAT | ENSG00000214941 | 26993 | RD |
| | | | TGATGGAGAAGAAACAAG | | | |
| 39 | NM_014481 | APEX2 | TTACACCACTTTCCACCTTCCTGTCCGAAGTACACGGACACT | ENSG00000169188 | 17889 | pCR |
| | | | AGCTGCCCCAGGAAGTTG | | | |
| 40 | NM_032195 | SON | ACTTGTGGGAAGGTAGTCCAAGGTACAACTATTTAGCTTCCC | ENSG00000159140 | 11183 | pCR |
| | | | GATTTGCTTCAAGGCTCT | | | |
| 41 | NM_152449 | LYSMD4 | AATCTCTGCAATAGGAAATGTTTCGATCATGCAAGTGCTTTC | ENSG00000183060 | 26571 | pCR |
| | | | CCAGCCAAATGTCTGTGC | | | |
| 42 | NM_018283 | NUDT15 | ACCATCTAATATCTGTATCATGCTATTAATAGGCTGTGATGT | ENSG00000136159 | 23063 | pCR |
| | | | TGAGTTTCTCACTTACTC | | | |
| 43 | NM_006341 | MAD2L2 | TGGATCCTGGCGGATGAGCAGGATGTCCACATGCATGACCCC | ENSG00000116670 | 6764 | pCR |
| | | | CGGCTGATACCACTAAAA | | | |
| 44 | NM_001291 | CLK2 | CAGACCTCAAGCCTGAAAATATTCTGTTTGTGAATTCAGACT | ENSG00000176444 | 2069 | pCR |
| | | | ATGAGCTCACCTACAACC | | | |
| 45 | NM_032941 | RECQL | GTGACAAAGTCCACGCAGAACTCTTTCAGGGCTGAATCGTCT | ENSG00000004700 | 9948 | pCR |
| | | | CAAACTTGTCATTCTGAA | | | |
| 46 | NM_017820 | EXD3 | AGGCCTGTAACTGTGACCAGTACCTAAAGGTCTCCAGGGACA | ENSG00000187609 | 26026 | RD |
| | | | TGATGAAGCAGCTCATGT | | | |
| 47 | NM_005293 | GPR20 | ACTGAAAACTCACAGGGGTAGCCGGGAGCTGCTCCTCACTTT | ENSG00000204882 | 4475 | pCR |
| | | | GGCCAGTTTGTCTCCAGA | | | |
| 48 | NM_198492 | CLEC4G | TAACCAAGGTTAGGTGACTGAGGACTGGAGCTGTTTGGTTTT | ENSG00000182566 | 24591 | pCR |
| | | | CTCGCATTTTCCACCAAA | | | |
| 49 | NM_005915 | MCM6 | TTGGTAGTTAACCCTAACTACTTGCTCGAAGATTGAGATAGT | ENSG00000076003 | 6949 | pCR |
| | | | GAAAGTAACTGACCAGAG | | | |
| 50 | NM_014034 | ASF1A | CCAGATGCAGATGCAGTAGGCGTAACTGTTGTGCTAATTACT | ENSG00000111875 | 20995 | pCR |
| | | | TGTACCTATCGAGGACAA | | | |
| 51 | NM_152633 | FANCB | ACAGTTATGTTCCAGTGCCTTCATAATCTCATCAGAATTCTC | ENSG00000181544 | 3583 | pCR |
| | | | CCTATAAACTGTTTCCTC | | | |
| 52 | NM_022836 | DCLRE1B | TCCAGGGGATTTAAAAGAGTTTTCTGCTTTGAGAGAGAAAT | ENSG00000118655 | 17641 | pCR |
| | | | AGAGAGTTTAGAAAGCAAT | | | |
| 53 | NM_018151 | RIF1 | ATGTATTCTTGGCTGCTATGCGTGGTTTTTCAGGAAATTTAA | ENSG00000080345 | 23207 | pCR |
| | | | TTATCTTACTGAGATGTG | | | |
| 54 | NM_005483 | CHAF1A | GTGCAGAGTTCTATATAGGATGCTGGATTAGTTCCTTTGATA | ENSG00000167670 | 1910 | pCR |
| | | | TTTGTAAAAATTCCCCCA | | | |
| 55 | NM_005484 | PARP2 | GGTCCGTATGCGGTACCTTTTAAAGGTTCAGTTTAATTTCCT | ENSG00000129484 | 272 | pCR |
| | | | TCAGCTGTGGTGAATGTT | | | |
| 56 | NM_002095 | GTF2E2 | GCTGTCAGTTTTCTGTGGATGAAGAATTTCAGAAACTGTGGA | ENSG00000197265 | 4651 | pCR |
| | | | GGAGTGTCACTGTAGATT | | | |
| 57 | NM_002691 | POLD1 | CTTGCAAGCATCCCATGGGGGGGGGGGGGGACCAGGGAGAAT | ENSG00000062822 | 9175 | pCR |
| | | | TAATAAAGTTCTGGACTT | | | |
| 58 | NM_004111 | FEN1 | CCTTAAGAGCTACAGCTAGAGAAACCTTCACAGGGGTGGAGA | ENSG00000168496 | 3650 | pCR |
| | | | GAGCATTCTAAGGTTTTT | | | |
| 59 | NM_194324 | TMSB15B | GGCAAGAGAGAAAGTGAATACGCTGATATAAGAAGCAAGAA | ENSG00000158164 | 30744 | pCR |
| | | | ACCTTCCTTCCTTCCAGAT | | | |
| 60 | NM_022362 | MMS19 | AGAACATAAGAGTCCATATTTCTAGTGGATTTGTAAAATAA | ENSG00000155229 | 13824 | RD |
| | | | GTGTGTGTGAGACACTTGC | | | |

### 4.4 Determining Expression Levels of Marker Genes

The determination of an expression level of one or more marker genes can be accomplished by any means known in the art such as Northern blotting, quantitative PCR (qPCR), microarray analysis or RNA-seq. Preferably, the expression levels of multiple marker genes are assessed simultaneously, by multiplex qPCR, microarray analysis or RNA-seq.

Microarray analysis involves the use of selected probes that are immobilized on a solid surface, termed an array. Said probes are able to hybridize to gene expression products such as mRNA, or derivates thereof such as cDNA. The probes are exposed to labeled gene expression products, or labelled derivates thereof such as labeled cDNA, hybridized, washed, after which the abundance of gene expression products or derivates thereof in the sample that are complementary to a probe is determined by determining the amount of label that remains associated to a probe. The probes on a microarray may comprise DNA sequences, RNA sequences, or copolymer sequences of DNA and RNA. The probes may also comprise DNA and/or RNA analogues such as, for example, nucleotide analogues or peptide nucleic acid molecules (PNA), or combinations thereof. The sequences of the probes may be full or partial fragments of genomic DNA. The sequences may also be in vitro synthesized nucleotide sequences, such as synthetic oligonucleotide sequences.

In the context of the invention, a probe preferably is specific for a gene expression product of a gene as listed in Table 1. A probe is specific when it comprises a continuous stretch of nucleotides that is complementary, over the whole length, to a nucleotide sequence of a gene expression product, or a cDNA product thereof. A probe can also be specific when it comprises a continuous stretch of nucleotides that is partially complementary to a nucleotide sequence of a gene expression product of said gene, or a cDNA product thereof. Partially means that a maximum of 5 nucleotides, more preferable 4 nucleotides, more preferable 3 nucleotides, more preferable 2 nucleotides and most preferable one nucleotide differs from the corresponding nucleotide sequence of a gene expression product of said gene. The term complementary is known in the art and refers to a sequence that is related by base-pairing rules to the sequence that is to be detected. It is preferred that the sequence of the probe is carefully designed to minimize nonspecific hybridization to said probe.

The specificity of probe is further determined by the hybridization and/or washing conditions. The hybridization and/or washing conditions are preferably stringent, which are determined by inter alia the temperature and salt concentration of the hybridization and washing conditions, as is known to a person skilled in the art. An increased stringency will substantially reduce non-specific hybridization to a probe, while specific hybridization is not substantially reduced. Stringent conditions include, for example, washing steps for five minutes at room temperature 0.1× sodium chloride-sodium citrate buffer (SSC)/0.005% Triton X-102. More stringent conditions include washing steps at elevated temperatures, such as 37° Celsius, 45° Celsius, or 65° Celsius, either or not combined with a reduction in ionic strength of the buffer to 0.05×SSC or even 0.01×SSC, as is known to a skilled person.

It is preferred that the probe is, or mimics, a single stranded nucleic acid molecule. The length of a probe can vary between 15 bases and several kilo bases, and is preferably between 20 bases and 1 kilobase, more preferred between 40 and 100 bases, and most preferred about 60 nucleotides. A most preferred probe comprises about 60 nucleotides. Said probe is preferably identical over the whole length to a nucleotide sequence of a gene expression product of a gene, or a cDNA product thereof. In a method of the invention, probes comprising probe sequences as indicated in Table 1 can be employed.

To determine an RNA expression level by micro arraying, gene expression products in the sample are preferably labeled, either directly or indirectly, and contacted with probes on the array under conditions that favor duplex formation between a probe and a complementary molecule in the labeled gene expression product sample. The amount of label that remains associated with a probe after washing of the microarray can be determined and is used as a measure for the gene expression level of a nucleic acid molecule that is complementary to said probe.

Image acquisition and data analysis can subsequently be performed to produce an image of the surface of the hybridized array. For this, the array may be dried and placed into a laser scanner to determine the amount of labeled sample that is bound to a probe at a predetermined spot. Laser excitation will yield an emission with characteristic spectra that is indicative of the labelled sample that is hybridized to a probe molecule.

An array preferably comprises multiple spots encompassing a specific probe. A probe preferably is present in duplicate, in triplicate, in quadruplicate, in quintuplicate, in sextuplicate or in octuplicate on an array. The multiple spots preferably are at randomized positions on an array to minimize bias. The amount of label that remains associated with a particular probe at each spot may be averaged, where after the averaged level can be used as a measure for the gene expression level of a nucleic acid molecule that is complementary to said probe. In addition, a gene product may be hybridized to two or more different probes that are specific for that gene product.

The determined RNA expression level can be normalized for differences in the total amounts of nucleic acid expression products between two separate samples by comparing the level of expression of one or more genes that are presumed not to differ in expression level between samples such as glyceraldehyde-3-phosphate-dehydro-genase, R-actin, and ubiquitin. Conventional methods for normalization of array data include global analysis, which is based on the assumption that the majority of genetic markers on an array are not differentially expressed between samples (Yang et al., 2002. Nucl Acids Res 30:15). Alternatively, the array may comprise specific probes that are used for normalization. These probes may detect RNA products from housekeeping genes such as glyceraldehyde-3-phosphate dehydrogenase and 18S rRNA levels, or a set of normalization such as provided in WO 2008/039071, which is hereby incorporated by reference, of which the RNA level is thought to be constant in a given cell and independent from the developmental stage or prognosis of said cell.

Another preferred method for determining RNA expression levels is by sequencing, preferably next-generation sequencing (NGS), of RNA samples, with or without prior amplification of the RNA expression products.

High throughput sequencing techniques for sequencing RNA, or RNA-seq, have been developed. NGS platforms, including Illumina® sequencing; Roche 454 Pyrosequencing®, ion torrent and ion proton sequencing, and ABI SOLiD® sequencing, allow sequencing of fragments of DNA in parallel. Bioinformatics analyses are used to piece these fragments together by mapping the individual reads. Each base is sequenced multiple times, providing high depth to deliver accurate data and an insight into unexpected DNA variation. NGS can be used to sequence a complete exome including all genes or, alternatively, to sequence a number of individual genes.

Pyrosequencing detects the release of inorganic pyrophosphate (PPi) as particular nucleotides are incorporated into the nascent strand (Ronaghi et al., 1996. Analytical Biochemistry 242: 84-9; Ronaghi, 2001. Genome Res 11: 3-11; Ronaghi et al., 1998. Science 281: 363; U.S. Pat. Nos. 6,210,891; 6,258,568; and 6,274,320, which are all incorporated herein by reference. In pyrosequencing, released PPi can be detected by being immediately conversion to adenosine triphosphate (ATP) by ATP sulfurylase, and the level of ATP generated is detected via luciferase-produced photons.

NGS also includes so called third generation sequencing platforms, for example nanopore sequencing on an Oxford Nanopore Technologies platform, and single-molecule real-time sequencing (SMRT sequencing) on a PacBio platform, with or without prior amplification of the RNA expression products.

Further high throughput sequencing techniques include, for example, sequencing-by-synthesis. Sequencing-by-synthesis or cycle sequencing can be accomplished by stepwise addition of nucleotides containing, for example, a cleavable or photobleachable dye label as described, for example, in U.S. Pat. Nos. 7,427,673; 7,414,116; WO 04/018497; WO 91/06678; WO 07/123744; and U.S. Pat. No. 7,057,026, all of which are incorporated herein by reference.

Sequencing techniques also include sequencing by ligation techniques. Such techniques use DNA ligase to incorporate oligonucleotides and identify the incorporation of such oligonucleotides and are inter alia described in U.S. Pat. Nos. 6,969,488; 6,172,218; and 6,306,597. Other sequencing techniques include, for example, fluorescent in situ sequencing (FISSEQ), and Massively Parallel Signature Sequencing (MPSS).

Sequencing techniques can be performed by directly sequencing RNA, or by sequencing a RNA-to-cDNA converted nucleic acid library. Most protocols for sequencing RNA samples employ a sample preparation method that converts the RNA in the sample into a double-stranded cDNA format prior to sequencing. Conversion of RNA into cDNA and/or cRNA using a reverse-transcriptase enzyme such as M-MLV reverse-transcriptase from Moloney murine leukemia virus, or AMV reverse-transcriptase from avian myeloblastosis virus, is known to a person skilled in the art.

Quantitative PCR (qPCR), or real-time PCR (RT-PCR), is a technique which is used to amplify and simultaneously quantify a template nucleic acid molecule such as an RNA. The detection of the amplification product can in principle be accomplished by any suitable method known in the art. The amplified products may be directly stained or labelled with radioactive labels, antibodies, luminescent dyes, fluorescent dyes, or enzyme reagents. Direct DNA stains include for example intercalating dyes such as acridine orange, ethidium bromide, ethidium monoazide or Hoechst dyes. These intercalating dyes are non-specific and bind to all double stranded DNA In the PCR. An increase in DNA products during amplification, results in an increased fluorescence intensity being measured. Another direct DNA detection method includes the use of sequence specific DNA probes consisting of a fluorescent reporter and quencher. Upon binding of the probe to its complementary sequence, polymerases of the PCR break the proximity of the reporter and the quencher, resulting in the emission of fluorescence. Commonly used reporter dyes include FAM (Applied Biosystems), HEX (Applied Biosystems), ROX (Applied Biosystems), YAK (ELITech Group) or VIC (Life Technologies) and commonly used quenchers include TAMRA (Applied Biosystems), BHQ (Biosearch Technologies) and ZEN (Integrated DNA Technologies). Alternatively, the amplified product may be detected by incorporation of labelled dNTP bases into the synthesized DNA fragments. Detection labels which may be associated with nucleotide bases include, for example, fluorescein, cyanine dye and BrdUrd.

For the simultaneous detection of multiple nucleic acid gene expression products, a multiplex qPCR can be used. In multiplex qPCRs, two or more template nucleic acid molecules are amplified and quantified in the same reaction. A commonly used method of achieving the simultaneously detection of multiple targets, is by using probes with different fluorescent dyes to distinguish distinct nucleic acid targets.

It is preferred in methods of the invention that genes are selected for normalization of the raw data. Preferred genes are genes of which the RNA expression levels are largely constant between individual samples comprising breast cancer cells from one individual, and between samples comprising breast cancer cells from different individuals. It will be clear to a skilled artisan that the RNA levels of said set of normalization genes preferably allow normalization over the whole range of RNA levels. An example of such a set of normalization genes is provided in WO 2008/039071, which is hereby incorporated by reference.

Normalization methods that may be employed include, for example, mean correction, linear combination of factors, Bayesian methods and non-linear normalization methods such quantile normalization. Preferred methods include non-parametric regression methods such as locally estimated scatterplot smoothing (LOESS; Jacoby, 2000. Electoral Studies 19: 577-613) and locally weighted scatterplot smoothing (LOWESS; Cleveland et al., 1988. J American Statistical Association 83: 596-610).

### 4.5 Prediction of an Individual's Response Following Auxiliary Immune Therapy

The invention provides a method for typing a sample to predict an individual's response following treatment with a poly ADP ribose polymerase (PARP) inhibitor. Typing of a sample can be performed in various ways. For example, the difference or similarity between a sample's expression profile and a previously established reference expression profile may be determined. The sample's expression profile is composed of the expression levels of a set of marker genes in said sample. The reference expression profile is composed of the average expression levels of the same set of marker genes in a sample from a reference group. The reference group may comprise a single individual. Preferably the reference group comprises the average expression levels of at least 10, 25, 50, 100, 200 or 300 individuals. The reference group may include individuals with different responses following treatment with a poly ADP ribose polymerase (PARP) inhibitor. The reference group may also include individuals that all show a positive response following treatment with a poly ADP ribose polymerase (PARP) inhibitor (i.e. response reference group) or individuals not showing a positive response following treatment with a poly ADP ribose polymerase (PARP) inhibitor (i.e. no response reference group). Alternatively, an expression profile of an individual can also be typed by comparing the individual's expression profile to multiple reference profiles. For example, the individual's expression profile can be compared to both reference profiles identified above (i.e. the response reference group and the no response reference group). If the expression profile of the individual's sample is substantially more similar to response reference group, when compared to the no response reference group, it will be predicted responsive.

The difference or similarity between an expression profile and one or more reference profiles can be determined by determining a correlation of the expression levels of marker genes in the profiles. For example, one can determine whether the expression levels of a subset of marker genes in a sample correlate to the expression levels of the same subset of marker genes in a reference profile. This correlation can be numerically expressed using a correlation coefficient. Several correlation coefficients can be used. Preferred methods are parametric methods which assume a normal distribution of the data. One of these methods is the Pearson product-moment correlation coefficient, which is obtained by dividing the covariance of the two variables by the product of their standard deviations.

Said correlations between the expression levels of marker genes in the individual's sample and the reference group, can be used to produce an overall similarity score for the set of marker genes used. A similarity score is a measure of the average correlation of gene expression levels of a set of genes in a sample from an individual with breast cancer and a reference profile. Said similarity score can, but does not need to be, a numerical value between +1, indicative of a high correlation between the gene expression level of the set of genes in a sample of said individual and said reference profile, and −1, which is indicative of an inverse correlation. A threshold can be used to differentiate between samples having a response, and samples having no response. Said threshold Is an arbitrary value that allows for discrimination between samples from individuals with no response, and samples of individuals with a response. If a similarity threshold value is employed, it is preferably set at a value at which an acceptable number of patients with a positive response would score as false negatives, and an acceptable number of patients with no positive response would score as false positives.

Based on the predictions made by the methods of the invention, one can determine a course of treatment of the individual with breast cancer. For example if the individual's expression profile is not substantially different from the no response group, or alternatively substantially different from the response group, this indicates that the individual is predicted to not show response following treatment with a poly ADP ribose polymerase (PARP) inhibitor. In that case, it is not recommended to provide said treatment with a poly ADP ribose polymerase (PARP) inhibitor.

Preferably, the predicted response following treatment with a poly ADP ribose polymerase (PARP) inhibitor is pCR. Alternatively, other responses to treatment with a poly ADP ribose polymerase (PARP) inhibitor could be assessed such as residual cancer burden (RCB), (3-year) event-free survival (EFS) and distant recurrence-free survival (DRFS).

A prediction of an individual's response following auxiliary immune therapy may be combined with other predictive or prognostic signatures, such as MAMMAPRINT® (U.S. Pat. Nos. 7,514,209 and 9,909,185, both of which are incorporated herein by reference), BLUEPRINT® (U.S. Pat. Nos. 9,175,351 and 10,072,301, both of which are incorporated herein by reference), OncotypeDX®, MapQuantDX™ ProSigna® and EndoPredict®, and/or with presence or absence of biomarkers such as Oestrogen Receptor (ER), Progesterone Receptor (PR) and Human Epidermal Growth factor Receptor 2 (HER2/ERBB2).

Said MAMMAPRINT analysis involves determining RNA expression levels of at least 5 genes selected from a list of 231 genes, such a set of 70 genes as depicted in van't Veer et al., 2002 (van't Veer et al., 2002. Nature 415: 530-536).

Said BLUEPRINT analysis involves determining RNA expression levels of at least adrenomedullin (ADM), Coiled-Coil Domain Containing 74B (CCDC74B), Moesin (MSN), Thrombospondin Type 1 Domain Containing 4 (THSD4), Perl-Like Domain Containing 1 (PERLD1) and Synaptonemal Complex Protein 3 (SYCP3), of Neuropeptide Y Receptor Y1 (NPY1R), SRY-Box Transcription Factor 11 (SOX11), ATP Binding Cassette Subfamily C Member 11 (ABCC11), Proline Rich 15 (PRR15) and Erb-B2 Receptor Tyrosine Kinase 2 (HER2; ERBB2), or of a combination thereof.

### 4.6 Methods of Treating an Individual with Breast Cancer

A method of treatment of breast cancer is usually determined based on the grade of the cancer, the stage of the cancer, the cancer's molecular subtype, or any combination thereof. The most common breast cancer molecular subtypes include breast cancers expressing a molecular target such as ER, progesterone receptor (PR) or HER2, and are classified as ER positive, HER2 positive, or triple negative, a breast cancer that lacks the expression of three molecular targets. As an alternative, or in addition, said classification may be based on the Luminal-type, HER2-type and Basal-type (Perou et al., 2000. Nature 406: 747-752; Krijgsman et al., 2012. Br Can Res Treatm 133: 37-47), for example by using the BluePrint signature as described in U.S. Pat. Nos. 9,175,351 and 10,072,301, both of which are incorporated herein by reference.

For a non-metastatic breast cancer, primary treatment may involve local treatment including surgery and often adjuvant post-operative radiotherapy. Surgery aims at the removal such a complete removal of the cancer tissue. In some instances, one or more of the axillary lymph nodes is removed as well.

Treatment of a nonmetastatic breast cancer may also involve systemic treatment depending on the molecular subtype of the breast cancer and is administered in addition to surgery. For hormone receptor positive (HR-positive; meaning ER and PR positive) breast cancer systemic treatment comprises endocrine therapy with or without chemotherapy. For HER2-positive breast cancer systemic therapy comprises chemotherapy combined with HER2-targeting therapy, by for example HER2-directed antibodies. For triple negative breast cancer, adjuvant therapy is mainly limited to chemotherapy.

The invention provides a method of typing a sample of an individual with breast cancer, said sample comprising breast cancer cells or comprising gene expression products from breast cancer cells, based on an expression profile of at least 7 genes from Table 1, whereby said typing is indicative of a response following treatment with a poly ADP ribose polymerase (PARP) inhibitor.

A PARP inhibitor preferably is selected from olaparib (3-aminobenzamide, 4-(3-(1-(cyclopropanecarbonyl)piperazine-4-carbonyl)-4-fluorobenzyl)phthalazin-1(2H)-one; AZD-2281; AstraZeneca), rucaparib (6-fluoro-2-[4-(methylaminomethyl)phenyl]-3,10-diazatricyclo[6.4.1.04,13]trideca-1,4,6,8(13)-tetraen-9-one; Clovis Oncology, Inc.); niraparib tosylate ((S)-2-(4-(piperidin-3-yl)phenyl)-2H-indazole-7-carboxamide hydrochloride; MK-4827; GSK); talazoparib (11S,12R)-7-fluoro-11-(4-fluorophenyl)-12-(2-methyl-1,2,4-triazol-3-yl)-2,3,10-triazatricyclo[7.3.1.05,13]trideca-1,5(13),6,8-tetraen-4-one; BMN-673; Pfizer); veliparib (2-[(2R)-2-methylpyrrolidin-2-yl]-1H-benzimidazole-4-carboxamide dihydrochloride benzimidazole carboxamide; ABT-888; Abbvie); pamiparib (2R)-14-fluoro-2-methyl-6,9,10,19-tetrazapentacyclo[14.2.1.02,6.08,18.012,17]nonadeca-1(18),8,12(17),13,15-pentaen-11-one; BGB-290; BeiGene); CEP-8983, and CEP 9722, a small-molecule prodrug of CEP-8983, a 4-methoxy-carbazole inhibitor (CheckPoint Therapeutics); E7016 (Eisai), PJ34 (2-(dimethylamino)-N-(6-oxo-5H-phenanthridin-2-yl)acetamide;hydrochloride) and 3-aminobenzamide.

A preferred PARP inhibitor is selected from the group consisting of olaparib, rucaparlb, niraparib, talazoparib, and pamiparib.

Said PARP inhibitor may be administered orally, as a tablet or as a capsule. Said PARP inhibitor preferably is administered once or twice per day for a period of 1-24 weeks, for example once or twice daily for a 12 weeks period. The preferred dosage of selected PARP inhibitors is 100-500 mg twice daily, preferably 300-400 mg twice daily for olaparib; 200-1000 mg twice daily, preferably 400-600 mg twice daily for rucaparib; 50-500 mg twice daily, preferably 100-300 mg twice daily for niraparib tosylate; 0.2-2 mg twice daily, preferably 0.5-1 mg twice daily for talazoparib; 100-600 mg twice daily, preferably 200-400 mg twice daily for veliparib; and 300-100 mg twice daily, preferably 40-60 mg twice daily for pamiparib. A person skilled in the art will understand that the dosage in a combination according to the invention, may be at the low range of the indicated dosages, or even below the indicated dosages.

The invention provides a method of treating an individual with breast cancer comprising typing the individual according to a method of the invention and treating an individual that is typed as having a response following treatment with a PARP inhibitor, with a PARP inhibitor, and treating an individual that is typed as not having a response following treatment with a PARP inhibitor with chemotherapy.

The invention provides a method of treating an individual with breast cancer comprising typing the individual according to the invention and treating the individual that is typed as having a response following treatment with a PARP inhibitor, with a combination of a PARP inhibitor and a platinum-based compound and/or a taxane such as cabazitaxel (Sanofi), docetaxel (Sanofi), paclitaxel (Celgene) and tesetaxel (Odonate Therapeutics), and treating the individual that is typed as not having a response following treatment with a PARP inhibitor with chemotherapy.

The Invention provides a use of a PARP Inhibitor for the treatment of an individual with breast cancer that Is typed according to the invention as having response following treatment with a PARP inhibitor.

The invention provides a use of a combination of a PARP inhibitor and a platinum-based compound for the treatment of an individual with breast cancer that is typed according to the invention as having response following treatment with a PARP inhibitor.

The invention further provides a use of a PARP inhibitor for the treatment of an individual with breast cancer that is typed according to the invention as having response following treatment with a PARP inhibitor, wherein said treatment further comprises a platinum-based compound.

The invention provides a PARP inhibitor for use in the treatment of an individual with breast cancer that is typed according to the invention as having response following treatment with a PARP inhibitor.

The invention provides a combination of a PARP inhibitor and a platinum-based compound for use in the treatment of an individual with breast cancer that is typed according to the invention as having response following treatment with a PARP inhibitor.

The invention further provides a PARP inhibitor for use in the treatment of an individual with breast cancer that is according to the invention as having response following treatment with a PARP inhibitor, wherein said treatment further comprises a platinum-based compound.

The invention provides a use of a PARP inhibitor in the preparation of a medicament for the treatment of an individual with breast cancer that is typed according to the invention as having response following treatment with a PARP inhibitor.

The invention provides a use of a combination of a PARP inhibitor and a platinum-based compound in the preparation of a medicament for the treatment of an individual with breast cancer that is typed according to the invention as having response following treatment with a PARP inhibitor.

The invention provides a use of a PARP inhibitor in the preparation of a medicament for the treatment of an individual with breast cancer that is typed according to the invention as having response following treatment with a PARP inhibitor, wherein said medicament further comprises a platinum-based compound.

Platinum-based compounds include cisplatin (Bristol Myers Squibb), carboplatin (Bristol Myers Squibb), oxaliplatin (Pfizer), nedaplatin (azanide;2-hydroxyacetic acid;platinum(2+)), satraplatin (Yakult Honsha), triplatin tetranitrate (azane;chloroplatinum(1+);hexane-1,6-diamine; platinum(2+);tetranitrate; Roche), phenanthriplatin (Blend Therapeutics), and Picoplatin (Poniard Pharmaceuticals).

Said platinum compound is preferably administered intravenously, preferably by infusion. For example, carboplatin may be administered at a dosage of 100-600 mg/m2, such as about 360 mg/m2, every 1-4 weeks and cisplatin may be administered at a dosage of 10-120 mg/m2, such as about 75 mg/m2, every 1-4 weeks.

An individual with breast cancer that is typed according to the invention as having no positive response following treatment with a PARP inhibitor, may be treated with a chemotherapeutic agent, not including a PARP inhibitor. Chemotherapeutic agents used in the treatment of individuals with cancer can be selected from following non-limiting examples: alkylating compounds such as bendamustine (Mundipharma Pharmaceuticals), busulfan (Pierre Fabre), carmustine (Bristol-Myers Squibb), chlorambucil (Aspen), cyclophosphamide (Baxter), dacarbazine (Pfizer), estramustine (Pfizer), ifosfamide (Baxter), lomustine (Kyowa Kirin Pharma), melphalan (GlaxoSmithKline), nimustine (Sankyo), procarbazine (Leadiant Biosciences), streptozotocin (Keocyt), temozolomide (Merck & Co), thiotepa (Adienne), treosulfan (Lamepro) and trofosfamide (Baxter); anthracyclines such as daunorubicin (Medac), doxorubicin (Pfizer), epirubicin (Pfizer), idarubicin (Pfizer), mitoxantrone (Pfizer), pirarubicin (Sanofi), pixantrone (Servier) and valrubicin (Endo Pharmaceuticals); anti-tumor antibiotics (not anthracyclines) such as bleomycin (Inovio Pharmaceuticals), dactinomycin (Ovation Pharmaceutical) and mitomycin (UroGen Pharma); antimetabolites such as azacitidine (Pfizer), capecitabine (Roche), cytarabine (Pfizer), cladribine (Janssen Pharmaceutica), clofarabine (Sanofi), decitabine (Janssen Pharmaceutica), fiudarabine (Bayer), (5-)fluorouracil (FivepHusion), 5-fluoro-2′-deoxyuridine (Sigma-Aldrich), gemcitabine (Eli Lilly and Company), (6-)mercaptopurin (Aspen), methotrexate (Aldeyra Therapeutics), nelarabine (Novartis), pemetrexed (Eli Lilly and Company), pentostatin (Pfizer) and (6-)tioguanine (Aspen); anti-mitotic cytostatics such as vinblastine (Teva), vincristine (Teva), vindesine (EG), vinfiunine (Pierre Fabre) and vinorelbine (Pierre Fabre), taxanes such as cabazitaxel (Sanofi), docetaxel (Sanofi), paclitaxel (Celgene) and tesetaxel (Odonate Therapeutics); non-taxane microtubule inhibitors such as eribulin (Eisai), indibulin (Baxter), ixabepilone (R-PHARM), patupilone (Novartis) and sagopilone (Bayer HealthCare); topo-isomerase inhibitors such as camptothecin (RTI International), etoposide (Bristol-Myers Squibb), irinotecan (Pfizer), teniposide (Bristol-Myers Squibb), tretinoin (Roche) and topotecan (Novartis); and histone deacetylase inhibitors such as chidamide (Chipscreen Bioscience, HLUYA Bioscience International), entinostat (Syndax), mocetinostat (Mirati therapeutics), tacedinaline (Pfizer), domatinostat (4SC), romidepsin (Celgene), abexinostat (Xynomic Pharmaceuticals), belinostat (Onxeo), nanatinostat (CHR-3996, Chroma Therapeutics, Viracta Therapeutics), givinostat (ITALFARMACO), MPTOE028 (3-(1-benzenesulfonyl-2,3-dihydro-1H-indol-5-yl)-N-hydroxy-acrylamide), panobinostat (Secura Bio Limited), pracinostat ((E)-3-[2-butyl-1-[2-(diethylamino)ethyl]benzimidazol-5-yl]-N-hydroxyprop-2-enamide), quisinostat (anssen Pharmaceuticals, NewVac), resminostat (4SC, Yakult Honsha), ricolinostat (Regenacy Pharmaceutica), trichostatin A (Vanda Pharmaceuticals), vorinostat (suberanilohydroxamic acid or SAHA, Merck & Co), butyric acid, 4-phenylbutyric acid, pivanex (pivaloyloxymethyl butyrate), valproic acid (2-propylpentanoic acid), cambinol (5-[(2-Hydroxynaphthalen-1-yl)methyl]-6-phenyl-2-thioxo-2,3-dihydropyrimidin-4(1H)-one), selistat (EX-527, AOP Orphan Pharmaceuticals AG), nicotinamide (pyridine-3-carboxamide) and sirtinol (2-[[(2-hydroxy-1-naphthalenyl)methylene]amino]-N-(1-phenylethyl)-benzamide).

Chemotherapy used in the treatment of individuals with breast cancer and typed according to the invention may comprises a therapeutically effective amount of any of the chemotherapeutic agents known to treat cancer patients. Said chemotherapeutic agent preferably includes a taxane, an anthracycline or alkylating compound. Said taxane preferably is paclitaxel or docetaxel. Said taxane is preferably administered Intravenously, preferably by infusion. Said taxane preferably is repeatedly administered, for example once every week once every two weeks, or once every three weeks. For example, paclitaxel may be administered at a dosage of 75-200 mg/m², such as about 80 mg/m², every 1-4 weeks; docetaxel may be administered at a dosage of 40-100 mg/m², such as about 60 mg/m², every 1-4 weeks; and cabazitaxel may be administered at a dosage of 5-75 mg/m², such as about 20 mg/m², every 1-4 weeks. Said anthracycline is preferably administered intravenously, preferably by infusion. Said anthracycline preferably is repeatedly administered, for example once every week, once every two weeks, or once every three weeks. Said anthracycline is preferably administered intravenously, preferably by infusion. For example, doxorubicin may be administered at a dosage of 20-400 mg/m2, such as about 60-75 mg/m2, every 1-4 weeks and epirubicin may be administered at a dosage of 20-140 mg/m2, such as about 60-90 mg/m2, every 1-4 weeks. Said alkylating compound is preferably cyclophosphamide. Said alkylating compound is preferably administered per oral or intravenously, preferably by infusion. Said alkylating compound preferably is repeatedly administered, for example once every week, once every two weeks, or once every three weeks. For example, cyclophosphamide may be administered at a dosage of 30-800 mg/m², such as about 600 mg/m², every 1-4 weeks. A person skilled in the art will understand that the dosage in a combination according to the invention, may be at the low range of the indicated dosages, or even below the indicated dosages.

Another preferred chemotherapy used in the treatment of individuals with breast cancer and typed according to the invention, comprises a combination of two or more chemotherapeutic agents. Examples of a preferred combination of chemotherapeutic agents include a combination of paclitaxel and carboplatin, a combination of paclitaxel and gemcitabine, a combination of doxorubicin and cyclophosphamide (often referred to as “AC”), a combination of doxorubicin, cyclophosphamide and paclitaxel (often referred to as “AC-P”), a combination of doxorubicin, cyclophosphamide and docetaxel (often referred to as “AC-T”), a combination of doxorubicin and docetaxel (often referred to as “AT”), a combination of cyclophosphamide, methotrexate and fluorouracil (often referred to as “CMF”), a combination of epirubicin, cyclophosphamide, methotrexate and fluorouracil (often referred to as “E-CMF”), a combination of epirubicin and cyclophosphamide (often referred to as “EC”), a combination of epirubicin, cyclophosphamide and paclitaxel (often referred to as “EC-P”), a combination of epirubicin, cyclophosphamide and docetaxel (often referred to as “EC-T”), a combination of fluorouracil, doxorubicin and cyclophosphamide (often referred to as “FAC” or “CAF”), a combination of fluorouracil, epirubicin and cyclophosphamide (often referred to as “FEC”), a combination of fluorouracil, epirubicin, cyclophosphamide and paclitaxel (often referred to as “FEC-P”), a combination of fluorouracil, epirubicin, cyclophosphamide and docetaxel (often referred to as “FEC-T”), a combination of docetaxel, doxorubincin and cyclophosphamide (often referred to as “TAC”) and a combination of docetaxel and cyclophosphamide (often referred to as “TC”). In said combination, chemotherapeutic agents are preferably administered intravenously, preferably by infusion. In said combination chemotherapeutic agents preferably are repeatedly administered, for example once every week, once every two weeks, or once every three weeks. In said combination chemotherapeutic agent have dosages preferably as follows: paclitaxel may be administered at a dosage of 75-200 mg/m², such as about 80 mg/m², every 1-4 weeks; carboplatin may be administered at a dosage of 100-300 mg/m2, such as about 300 mg/m², every 1-4 weeks; carboplatin may be administered at a dosage of 100-300 mg/m2, such as about 300 mg/m², every 1-4 weeks; gemcitabine may be administered at a dosage of 500-3500 mg/m², such as about 1250 mg/m², every 1-4 weeks; cyclophosphamide may be administered at a dosage of 30-800 mg/m2, such as about 600 mg/m2, every 1-4 weeks; methotrexate may be administered at a dosage of 10-100 mg/m², such as about 40 mg/m², every 1-4 weeks; doxorubicin may be administered at a dosage of 10-100 mg/m², such as about 50 mg/m², every 1-4 weeks; fluorouracil may be administered at a dosage of 100-4000 mg/m², such as about 500 mg/m², every 1-4 weeks; epirubicin may be administered at a dosage of 50-200 mg/m², such as about 100 mg/m². In said combination methotrexate and cyclophosphamide, can be administered per oral or intravenously.

For the purpose of clarity and a concise description, features are described herein as part of the same or separate aspects and preferred embodiments thereof, however, it will be appreciated that the scope of the invention may include embodiments having combinations of all or some of the features described.

The invention will now be illustrated by the following examples, which are provided by way of illustration and not of limitation and it will be understood that many variations in the methods described and the amounts indicated can be made without departing from the spirit of the invention and the scope of the appended claims.

### 5 EXAMPLES

### Example 1: Identification and Performance of 60 Marker Genes for the Prediction of pCR Following

### Materials and Methods

### Sample Collection and Processing

For this study, 72 FF pre-treatment biopsies collected from patients treated with Paclitaxel+Veliparib+Carboplatin (VC arm)[Rugo et al., 2016. N Engl J Med 375: 23-34] within the ISPY2 trial (ADD TRIAL ID), were analyzed with full-genome microarray (amadid 15746) at Agendia The clinical data included: pathological complete response status (either pCR or residual disease (RD), hormone receptor (HR) status and molecular subtyping obtained with BluePrint. Table 2 and Table 3 report patient sample distribution based on response status, HR, BluePrint and MammaPrint results.

To translate the index obtained on fast frozen (FF) samples to an index obtained with the same templates applied on formalin fixed paraffin embedded (FFPE) tissue expression, a set of 74 matched FF-FFPE samples so-called internally at Agendia as Quantum Leap (QL) dataset, was used.

Also, to translate the signature from amadid #15746, to the current amadid #32627, 21 paired ISPY2 samples were used.

TABLE 2
Description of the ISPY2 VC arm.

| Group | Subgroup | N |
|---|---|---|
| Response status | pCR | 45 |
| | RD | 27 |
| Hormone Receptor Status | HR+ | 33 |
| | HR− | 39 |
| BluePrint | Luminal | 23 |
| | Basal | 49 |
| | HER2 | 0 |
| MammaPrint | High Risk | 72 |
| | Low Risk | 0 |

TABLE 3
The distribution of HR status and BluePrint subtypes for
responders (pCR) and non-responders (RD) in the VC arm.

| | |
|---|---|
| HR status | BluePrint |

| | HR+ | HR− | Luminal | Basal |
|---|---|---|---|---|
| pCR | 5 | 22 | 0 | 27 |
| RD | 28 | 17 | 23 | 22 |

### Validation Data

### BrighTNess

For validation purposes BrighTNess dataset was used [Loibl et al., 2018. Lancet Oncol 19: 497-509; Metzger-Filho et al., 2021. NPJ Breast Cancer 7: 142]. BrighTNess was a phase III, multicenter, randomized, double-blind, placebo-controlled study that enrolled stage II/III TNBC patients to receive neoadjuvant chemotherapy with paclitaxel followed by doxorubicin/cyclophosphamid (AC), or the same plus Carboplatin or Carboplatin plus the PARPi Veliparib (i.e. ABT 888) concurrent with paclitaxel. Whole transcriptome RNA sequencing (RNAseq) was performed on pre-treatment FF biopsies. The details on the patient selection for this trial and the outcome can be found in [Loibl et al., 2018. Lancet Oncol 19: 497-509]. The preprocessed data was obtained from Gene Omnibus (GSE164458). Raw gene counts were not available for this cohort.

The extraction protocol was as follows. Pre-treatment biopsies were collected in RNAlater (ThermoFisher Scientific, Waltham, MA USA) and total RNA was extracted. Total RNA underwent whole transcriptome RNA sequencing (RNAseq) using RiboZero Gold rRNA (Illumina, San Diego, CA USA) depletion. All samples were sequenced on an Illumina HiSeq 3000 with single-end 50 bp reads.

The preprocessing of the data was performed as follows. RNA-seq reads were aligned to the Ensembl release 76 top-level assembly with STAR version 2.0.4b [Dobin et al., 2013. Bioinformatics 29: 15-21]. Gene counts were derived from the number of uniquely aligned unambiguous reads by Subread: featureCount version 1.4.5 [Liao et al., 2014. Bioinformatics 30: 923-930]. Samples with >10 million unique reads were included for further analyses. Transcript counts were produced by Sailfish version 0.6.3 [Patro et al., 2014. Nature Biotech 32: 462-464]. Sequencing performance was assessed for total number of aligned reads, total number of uniquely aligned reads, genes and transcripts detected, ribosomal fraction known junction saturation and read distribution over known gene models with RSeQC version 2.3 [Wang et al., 2012. Bioinformatics 28: 2184-5]. Genome_build: Ensembl release 76 [Zerbino et al., 2015. Genome Biol 16: 56]. Data was log 2 normalized and the normalized expressions uploaded on Gene Omnibus. Platform id GPL21290.

The response by demographic characteristics is presented in Loibl et al., 2018. Lancet Oncol 19: 497-509.

For the stability of the signature over time, the FFPE controls from MammaPrint were used. More specifically, PBCL2 (BluePrint Luminal; MammaPrint Low Risk), PHHE2 (BluePrint HER2, MammaPrint High Risk), PHTR2 (BluePrint Basal, MammaPrint High Risk) and PLEP3 (BluePrint Luminal, MammaPrint Low Risk). The controls amounted to 372, 448, 723 and 503 data points respectively with a time span between October 2018 and June 2020, for PBCL2, PHTR2 and PLEP3, and between September 2019 and June 2020 for PHHE2 (as the latter is a newer control sample).

For the prevalence, both veliparib dataset described previously and 3835 FFPE 44K samples from FLEX studies (clinicaltrials.gov: NCT03053193) were used. The distribution of HR status and BluePrint results in the FLEX samples is as follows: 267 HR−HER2− (TN) and 3568 HR+HER2, 3436 Luminal-type, 372 Basal-type with no HER2-type samples included.

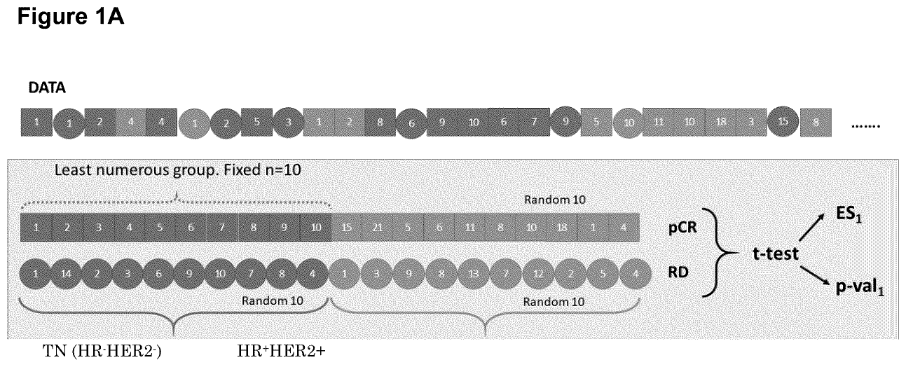

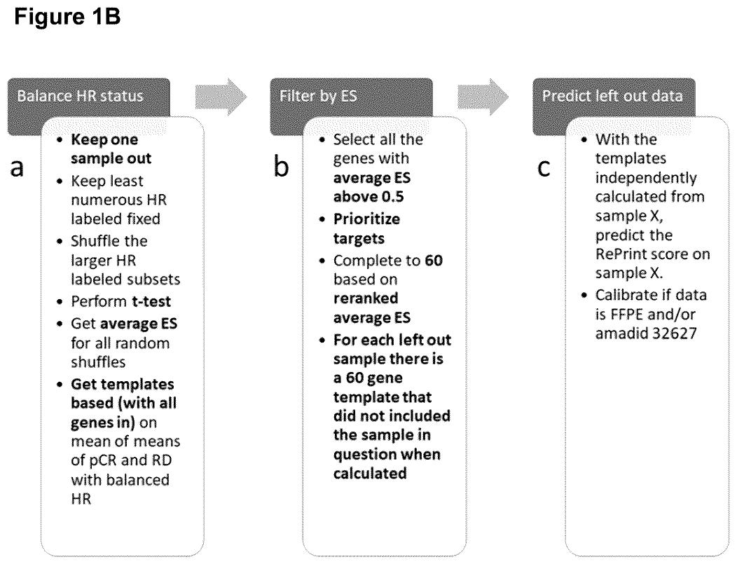

The development of the RePrint gene signature has at its core the leave-one-out cross-validation (LOOCV) [Mining, 2009. Math Intell 27: 83-85] which was used on the VC dataset. More specifically on one iteration of LOOCV, one sample was left for validation and 71 remaining others were used for differentially expressed analysis (DEA) and template training (FIG. 1A). The DEA was performed between pCR and RD and in each balancing the HR status (FIG. 1A). From Table 3 it is evident that the least numerous HR status for pCR is HR+HER2− with n=5, thus 10 samples in pCR were selected (5 HR+HER2− and 5 TN) and the same numbers (5 HR+HER2− and 5 TN) were in the RD group. As the TN samples for pCR group are more numerous, a sequential shuffling was applied with a random selection of 5 at each shuffle. For one LOOCV iteration, 100 DEA comparisons with 5 randomly selected samples in each HR status were performed. Due to small sample size in each group (n=10), a t-test was used for the DEA. For each DEA, the effect size (ES) and p-value was calculated, for each gene in the array. The variables (i.e. the probes) were sorted according to the average ES (averaged over the random shuffles within one LOOCV iteration). Only probes that have average ES>0.5 were further used.

Alongside the statistical significance, of key importance is the biological functions of the genes. Thus, a list of biologically relevant genes, i.e. genes relevant to a DRD phenotype (also named DRD+), was compiled and used as an additional criterion for gene selection. All genes from the biologically relevant gene list that had average ES>0.5 [Sullivan and Feinn, 2012. J Grad Med Educ 4: 279-282] were included in the signature (FIG. 1B). The rest of the genes that passed the ES filtering step but were not in the list, were sorted by the average ES and added one by one completing the signature to 60 genes.

The signature was validated on the discovery set using LOOCV strategy described above.

For the validation on BrighTNess, the VC arm data were quantile normalized and standardized (subtracting the mean and dividing by the standard deviation for each gene) to calculate the templates. Further, the BrighTNess data was also standardized. The standardized templates were applied on the standardized BrighTNess data to calculate the RePrint index using the equations described above. The performance of both validation methods was assessed using accuracy, sensitivity and specificity metrics. The significance of the depending variables in BrighTNess data was estimated using multinomial logistic regression from statsmodels library [Seabold and Perktold, 2010. Proc. 9th Python Sci. Conf., Scipy. 57: 92-96].

### RESULTS AND DISCUSSION

Using the LOOCV described in the methods section, 2861 significant genes (average ES>0.5 FIG. 1, step b) were initially identified. From these, 40 genes were in the compiled biologically relevant gene list (data not shown). These 40 genes are defining the backbone of the signature. The signature was completed with 20 other highly significant genes (based on ranked average ES) that ultimately yielded high accuracy, sensitivity and specificity when used in a LOOCV algorithm.

The list of the 60 genes which define the RePrint signature and their annotations are shown in Table 1.

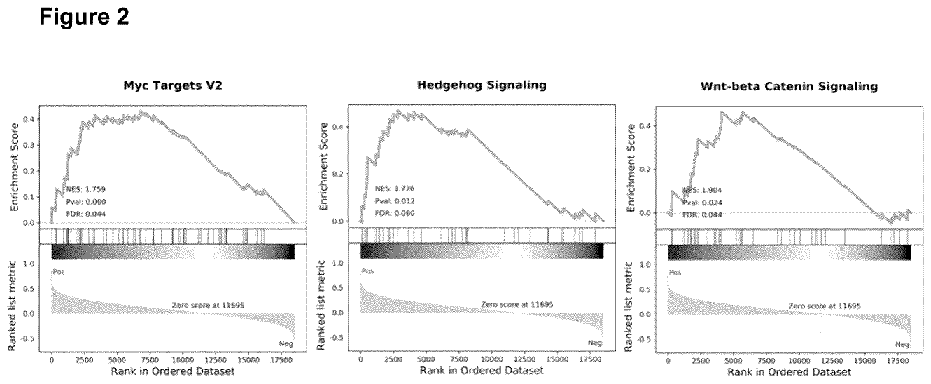

Pathway analysis (see FIG. 2 and Table 4) and DAVID functional annotation clustering [Sherman et al., 2022. Nucleic Acids Research: 10.1093/nar/gkac194] showed the biological relevance of the RePrint 60 genes.

TABLE 4
Pathway analysis for the RePrint 60 gene signature

| Pathways | Gene |
|---|---|
| Homologous recombination | SMC6, BRCA1, SWS1 |
| Non-homologous end-joining | XRCC6 |
| Mismatch excision repair | MSH2, MSH3, MSH6 |
| Nucleotide excision repair | GTF2E2 |
| Base excision repair (BER) | MUTYH |
| Fanconi anemia | FANCB, FANCE, FANCC, |
| | FANCA, BRIP1 |
| DNA polymerases | MAD2L2, POLD1 |
| Ubiquitination and modification | USP1, RNF168, RNF8 |
| PARP enzymes that bind to DNA | MGMT, ALKBH3, PARP2 |
| Chromatin Structure and Modification | CHAF1A, H2AFX |
| Editing and processing nucleases | DCLRE1B, EXO1, FEN1 |
| Other conserved DNA damage response | CLK2, RIF1, RAD17, |
| genes | RAD9A, PCNA |
| Other BER and strand break joining | APEX1, APEX2, LIG3, |
| factors | PER1 |

The contingency table (Table 5) of the VC arm cohort indicates that RePrint predicts pCR with accuracy of 94.44%, sensitivity of 96.29% and specificity of 93.33%. High sensitivity alleviates the cost of not receiving the drug, when in reality the patients would benefit from the treatment which indicates the high safety and reliability of the signature.

TABLE 5
Contingency table of the entire ISPY2 VC arm.

Predicted

| | | RD (DRD−) | pCR (DRD+) |
|---|---|---|---|
| Actual | RD | 42 | 3 |
| | pCR | 1 | 26 |

When splitting on HR status, RePrint still accurately predicted response. The proportions of true positive, true negatives, false positives and false negatives for each group (HR+HER2− and Triple Negative (TN)) are shown in the contingency tables Table 6 and Table 7. The accuracy, sensitivity and specificity for the TN patients were 94.87%, 95.45% and 94.11%, respectively. For the HR+HER2− patients the accuracy, sensitivity and specificity are 93.93%, 100% and 92.85%, respectively. In both cases the signature yielded high sensitivity.

TABLE 6
Contingency table of the TN patients
from the ISPY2 VC arm treated cohort.

Predicted

| | | |
|---|---|---|
| TN (HR− HER2−) | RD (DRD−) | pCR (DRD+) |

| | | | |
|---|---|---|---|
| Actual | RD | 16 | 1 |
| | pCR | 1 | 21 |

TABLE 7
Contingency table of the HR+HER2− patients
from the ISPY2 VC arm treated cohort.

Predicted

| | | |
|---|---|---|
| TN (HR+ HER2−) | RD (DRD−) | pCR (DRD+) |

| | | | |
|---|---|---|---|
| Actual | RD | 26 | 2 |
| | pCR | 0 | 5 |

A clustered heatmap of the VC cohort (data not shown) using the RePrint 60 genes showed that there is a clear separation between the pCR and RD samples when the 60 gene signature is used indicating the high predictive power of RePrint. It should be noticed that some degree of overfitting might occur, given that the dataset used for the development was employed for multiple training steps. A more objective view is when using an independent dataset which is similarly treated (i.e. PARPi and Carboplatin).

One of the available independent validation sets for RePrint was the BrighTNess trial dataset (see Materials and methods section for details). It should be noted that, although the patients were treated with a combination of veliparib and carboplatin, which is suitable for RePrint validation, the actual dataset was generated using an NGS platform and exhibits very low intensities after the preprocessing. For the validation process, each arm was evaluated separately as well as the combination between arms A and B (see details in BrighTNess sub-section of the Materials and methods section) as there was no substantial difference between only carboplatin (Arm B) and veliparib+carboplatin (Arm A) in pCR rates.

The accuracy, sensitivity and specificity of arm A were 59%, 57% and 61% and for the arm B 59%, 63% and 57%, respectively (data not shown). The 60 gene signature on BrighTNess data (Arms A and B combined) had an accuracy of 59%, sensitivity of 58% and specificity of 60% (data not shown). A significance test performed on the distribution of RePrint indices per response for arms A, B and A+B were indicating a significant difference (data not shown). The control arm, arm C, yielded an accuracy of 57% with the sensitivity, and specificity of 53% and 59%, respectively (data not shown).

Another important aspect of the BrighTNess dataset is that the response may be dependent on the lymph node status (Loibl et al., 2018. Lancet Oncol 19: 497-509). The multinomial logistic regression seems to support this hypothesis (data not shown).

When only lymph node negative patients were evaluated for DRD status using RePrint signature, the performance increases. Namely, the accuracy raised to 61%, sensitivity to 58.3% and specificity to 65% (data not shown).

A prevalence study is important for clinical utility of the signature. Relatively high prevalence indicates that a considerable large population may benefit from the test. The prevalence was performed on the FLEX pool of data (see herein above). The dataset comprises 267 TN (HR−HER2−) samples of which 135 are DRD+, 3568 HR+HER2− samples of which 85 are DRD+. The distribution of BluePrint subtypes are as follows: 3436 Luminal-type, 372 Basal-type samples. The results of the prevalence for the FLEX dataset are shown in Table 8.

Approximately 60% of the TN patients are DRD+, which means that these patients potentially can benefit from a PARP inhibitor treatment, optionally combined with platinum-based treatment. This is in line with the previously reported percentages in the literature [Wolf et al., 2017. NPJ Breast Cancer 3: 1-8, 2017].

TABLE 8
Prevalence of DRD positive in the FLEX pool.

| DRD+ | | | |
|---|---|---|---|
| FLEX | Basal | Luminal | Total |
| HR+ | 83 | 2 | 85 |
| HER2− | (62.40%) | (0.06%) | (2.41%) |
| TN | 135 | 0 | 135 |
| | (50.56%) | | (50.56%) |
| Total | 218 | 2 | 221 |
| | (58.60%) | (0.06%) | (5.76%) |

From the data used for the development (veliparib/carboplatin—VC), we observe similar percentages in TN patients indicating the consistency of the prevalence. The cohort comprises 39 TN (HR−HER2−) samples of which 22 are DRD+(56%) and 33 HR+HER2− samples of which 7 DRD+(20%). The BluePrint subtype distribution is as follows: 23 Luminal-type samples and 49 Basal-type (40 MP high-risk).

There is a large overlap between DRD+ patients and immune sensitivity, as estimated with ImPrint. In the FLEX cohort, 75% of the DRD+ individuals are also typed as being immune sensitive.

1. A method of typing a sample of an individual with breast cancer, said sample comprising breast cancer cells or comprising gene expression products from breast cancer cells, the method comprising:
(i) isolating RNA from the sample obtained from the individual;
(ii) determining an expression level of at least 7 marker genes to thereby provide an expression profile of the marker genes, wherein the marker genes are selected from the genes listed in Table 1;
(iii) comparing the individual's expression profile to a reference expression profile of the at least 7 marker genes;
thereby typing the sample for a response following treatment with a poly ADP ribose polymerase (PARP) inhibitor.

2. The method according to claim 1, wherein the ample is either a fresh frozen or a formalin-fixed paraffin-embedded sample.

3. The method according to claim 1, wherein an individual who is typed as positively responding to a PARP inhibitor is treated with a PARP inhibitor.

4. The method according to claim 1, wherein treatment with a PARP inhibitor is combined with a platinum-based compound.

5. The method according to claim 1, wherein the determination of the expression profile is performed using RNA-sequencing or microarray gene expression analysis.

6. The method according to claim 1, wherein the expression profile comprises at least 8 different marker genes, 10 different marker genes, preferably at least 20 different marker genes, more preferably at least 30 different marker genes, more preferably at least 40 different marker genes, more preferably at least 50 different marker genes selected from the genes listed in Table 1, most preferably all marker genes listed in Table 1.

7. The method according to claim 1, wherein the reference expression profile is composed of the average expression levels of the marker genes specified in step (ii) of individuals having a positive response to a PARP inhibitor, of individuals not having a positive response to a PARP inhibitor; or of a mixture of individuals having a positive response to a PARP inhibitor and individuals not having a positive response to a PARP inhibitor.

8. The method according to claim 1, wherein the individual's expression profile is compared to two reference expression profiles, wherein one reference expression profile is composed of the average expression levels of the marker genes specified in step (ii) of individuals having a positive response to a PARP inhibitor and the other reference expression profile is composed of the average expression level of the marker genes specified in step (ii) of individuals not having a positive response to a PARP inhibitor.

9. The method according to claim 1, wherein the response is a pathologic complete response (pCR).

10. A method of treating an individual with breast cancer, comprising:
typing of a sample from said individual using the method according to claim 1;
treating the individual that is typed as having a positive response to a PARP inhibitor with a PARP inhibitor, optionally in combination with a platinum-based compound and/or a taxane; and
treating the individual that is typed as not having a positive response to a PARP inhibitor with chemotherapy, immunotherapy, or a combination thereof.

11. The method according to claim 10, wherein the PARP inhibitor comprises veliparib.

12. The method according to claim 10, wherein the PARP inhibitor comprises olaparib.

13. The method according to claim 10, wherein the PARP inhibitor comprises talazoparib.

14. The method of claim 10, wherein the PARP inhibitor is combined with a platinum-based compound.

15. The method according to claim 14, wherein the platinum-based compound comprises carboplatin.


SEQUENCE LISTING (60 sequences)

SEQ ID NO: 1 - DNA, 60 bp, synthetic construct
  source 1..60: mol_type = other DNA
aaactagcagatgttcctggagaaattttgaagaaagcagctcacaagtcaaaagagctg

SEQ ID NO: 2 - DNA, 60 bp, synthetic construct
  source 1..60: mol_type = other DNA
aagcatccagaagagaaagcatcataatgccgagaacaagccggggttacagatcaaact

SEQ ID NO: 3 - DNA, 60 bp, synthetic construct
  source 1..60: mol_type = other DNA
tcctttcagatgcctggatgaatttgatgtctacatggatatggttaataggagaattgc

SEQ ID NO: 4 - DNA, 60 bp, synthetic construct
  source 1..60: mol_type = other DNA
gaatctgaccttactctagcaggagtttttgtagactctccctgatagtttagtttttga

SEQ ID NO: 5 - DNA, 60 bp, synthetic construct
  source 1..60: mol_type = other DNA
gaagtttgcctggctagtgaaaggtcaactgtagatgctgaagctgtccataaattgctg

SEQ ID NO: 6 - DNA, 60 bp, synthetic construct
  source 1..60: mol_type = other DNA
atgagggcctcgtttattaagatctttaaactgctttatacactgtcacgtggcttcatc

SEQ ID NO: 7 - DNA, 60 bp, synthetic construct
  source 1..60: mol_type = other DNA
agggcggccctgtactttttgtggcttggtttctctggaaatgtcaccttttcgggcgca

SEQ ID NO: 8 - DNA, 60 bp, synthetic construct
  source 1..60: mol_type = other DNA
ggaaatctgccactaaccttccccaactcaaggaactgtaccagttgtccaaggagaagg

SEQ ID NO: 9 - DNA, 60 bp, synthetic construct
  source 1..60: mol_type = other DNA
cgcaagatgaaatttgctctgctaaaaggctccttcagtgagcaaggcatcaacgagttt

SEQ ID NO: 10 - DNA, 60 bp, synthetic construct
  source 1..60: mol_type = other DNA
gtgagtggtttaaggagcggtcagtgtgtataaagtgtgtttgaacattatgccaaatat

SEQ ID NO: 11 - DNA, 60 bp, synthetic construct
  source 1..60: mol_type = other DNA
aagaaaggattagattgggtaaaggaagaagccccagatatactgtgccttcaagagacc

SEQ ID NO: 12 - DNA, 60 bp, synthetic construct
  source 1..60: mol_type = other DNA
ttgggcatggactaatttgtatctgtttaactcatattctgcacgatctgtatatagtac

SEQ ID NO: 13 - DNA, 60 bp, synthetic construct
  source 1..60: mol_type = other DNA
acactgtcctggtgcccagcccaacgtggtccaacgtttttttataataaagtcctcgtt

SEQ ID NO: 14 - DNA, 60 bp, synthetic construct
  source 1..60: mol_type = other DNA
agaattccagtcttgagaaacaggtcaaggacagcttcaaaagagattctaaataaatgt

SEQ ID NO: 15 - DNA, 60 bp, synthetic construct
  source 1..60: mol_type = other DNA
tttatgtttttgaggctttctgttgccatggtgatggtgtagccctcccactttgctgtt

SEQ ID NO: 16 - DNA, 60 bp, synthetic construct
  source 1..60: mol_type = other DNA
ttactctgcaatctttatcgcaatgagaaggacagcgtggactggcacagtgatgatgaa

SEQ ID NO: 17 - DNA, 60 bp, synthetic construct
  source 1..60: mol_type = other DNA
tgctgctgtttcagccgcagctgaatttgagaatgctgaatctgaggctgatttcagtat

SEQ ID NO: 18 - DNA, 60 bp, synthetic construct
  source 1..60: mol_type = other DNA
atgacctagctgaaactgatagaggaaatagattggctaacaattcagattgcattttag

SEQ ID NO: 19 - DNA, 60 bp, synthetic construct
  source 1..60: mol_type = other DNA
caagttttccttgctttcctgatactctttggcgctgacttggaattctaagagccttgg

SEQ ID NO: 20 - DNA, 60 bp, synthetic construct
  source 1..60: mol_type = other DNA
aaccagatcagaaagcagcagtcaagctgatactagcaaatccaaatcagaagatagtgc

SEQ ID NO: 21 - DNA, 60 bp, synthetic construct
  source 1..60: mol_type = other DNA
aagtgtttttcataaacccattatccaggactgtttatagctgttggaaggactaggtct

SEQ ID NO: 22 - DNA, 60 bp, synthetic construct
  source 1..60: mol_type = other DNA
aacggctgctatttgaactattacttttttcttctggctgctattcaaggagctaccctc

SEQ ID NO: 23 - DNA, 60 bp, synthetic construct
  source 1..60: mol_type = other DNA
tctgctcctactgtatcaatgaatggatgaagcggaagatagaatgccccatttgtcgga

SEQ ID NO: 24 - DNA, 60 bp, synthetic construct
  source 1..60: mol_type = other DNA
aggacctgtttaaccagaaagataagactgttcttaacgacctagatgattctgttcatc

SEQ ID NO: 25 - DNA, 60 bp, synthetic construct
  source 1..60: mol_type = other DNA
tcattatttgggtaggtgacctgcttggatctagacagcatctgttcaaagttgtagggc

SEQ ID NO: 26 - DNA, 60 bp, synthetic construct
  source 1..60: mol_type = other DNA
cactccctacagcctagcatttcacagaaaagtgtttttcagatgtttcagagatgcaca

SEQ ID NO: 27 - DNA, 60 bp, synthetic construct
  source 1..60: mol_type = other DNA
ctagaaccttaggcattggggagttttagatggactaattttattaaaggattgtttttt

SEQ ID NO: 28 - DNA, 60 bp, synthetic construct
  source 1..60: mol_type = other DNA
gggtaccagtgggctcagatgaagctcttgtgctcatgtatattatgaccttctgtgttt

SEQ ID NO: 29 - DNA, 60 bp, synthetic construct
  source 1..60: mol_type = other DNA
ccacatgcgagtattaataaataaagacaccattggcctgacgcagctccctcaaactct

SEQ ID NO: 30 - DNA, 60 bp, synthetic construct
  source 1..60: mol_type = other DNA
ctttgaggaagcggttcgaagagttcttgctaccgaggataggtcagatcatttgattca

SEQ ID NO: 31 - DNA, 60 bp, synthetic construct
  source 1..60: mol_type = other DNA
accccttgttgtagagtataaaattgcggatatgggacacttaaaatactacttggctcc

SEQ ID NO: 32 - DNA, 60 bp, synthetic construct
  source 1..60: mol_type = other DNA
atgaaggtggtgtgttttttctggatatcacattttcatcagattatccatttaagccac

SEQ ID NO: 33 - DNA, 60 bp, synthetic construct
  source 1..60: mol_type = other DNA
atgggccagcaagtcctggataatttctttcggtctcacatctccactgatgcacacagc

SEQ ID NO: 34 - DNA, 60 bp, synthetic construct
  source 1..60: mol_type = other DNA
aaccctttttccatagtgttaactgtcagtgcccatgggctatcaacttaataagatatt

SEQ ID NO: 35 - DNA, 60 bp, synthetic construct
  source 1..60: mol_type = other DNA
ttgactccttttcaaccatttgtcacagacgtgagaagaagaaatgacttcaaaatcaag

SEQ ID NO: 36 - DNA, 60 bp, synthetic construct
  source 1..60: mol_type = other DNA
ttctcagaactcagatgtggctagaccaaccagtgggaaactgccccagcttctcccacc

SEQ ID NO: 37 - DNA, 60 bp, synthetic construct
  source 1..60: mol_type = other DNA
tcttcaccatcccgttttccagcaagagtcgttcaccagacaggtgttatggaagctgct

SEQ ID NO: 38 - DNA, 60 bp, synthetic construct
  source 1..60: mol_type = other DNA
agcagctaagtgtctctgacaagcagttgactgacatattattgatggagaagaaacaag

SEQ ID NO: 39 - DNA, 60 bp, synthetic construct
  source 1..60: mol_type = other DNA
ttacaccactttccaccttcctgtccgaagtacacggacactagctgccccaggaagttg

SEQ ID NO: 40 - DNA, 60 bp, synthetic construct
  source 1..60: mol_type = other DNA
acttgtgggaaggtagtccaaggtacaactatttagcttcccgatttgcttcaaggctct

SEQ ID NO: 41 - DNA, 60 bp, synthetic construct
  source 1..60: mol_type = other DNA
aatctctgcaataggaaatgtttcgatcatgcaagtgctttcccagccaaatgtctgtgc

SEQ ID NO: 42 - DNA, 60 bp, synthetic construct
  source 1..60: mol_type = other DNA
accatctaatatctgtatcatgctattaataggctgtgatgttgagtttctcacttactc

SEQ ID NO: 43 - DNA, 60 bp, synthetic construct
  source 1..60: mol_type = other DNA
tggatcctggcggatgagcaggatgtccacatgcatgacccccggctgataccactaaaa

SEQ ID NO: 44 - DNA, 60 bp, synthetic construct
  source 1..60: mol_type = other DNA
cagacctcaagcctgaaaatattctgtttgtgaattcagactatgagctcacctacaacc

SEQ ID NO: 45 - DNA, 60 bp, synthetic construct
  source 1..60: mol_type = other DNA
gtgacaaagtccacgcagaactctttcagggctgaatcgtctcaaacttgtcattctgaa

SEQ ID NO: 46 - DNA, 60 bp, synthetic construct
  source 1..60: mol_type = other DNA
aggcctgtaactgtgaccagtacctaaaggtctccagggacatgatgaagcagctcatgt

SEQ ID NO: 47 - DNA, 60 bp, synthetic construct
  source 1..60: mol_type = other DNA
actgaaaactcacaggggtagccgggagctgctcctcactttggccagtttgtctccaga

SEQ ID NO: 48 - DNA, 60 bp, synthetic construct
  source 1..60: mol_type = other DNA
taaccaaggttaggtgactgaggactggagctgtttggttttctcgcattttccaccaaa

SEQ ID NO: 49 - DNA, 60 bp, synthetic construct
  source 1..60: mol_type = other DNA
ttggtagttaaccctaactacttgctcgaagattgagatagtgaaagtaactgaccagag

SEQ ID NO: 50 - DNA, 60 bp, synthetic construct
  source 1..60: mol_type = other DNA
ccagatgcagatgcagtaggcgtaactgttgtgctaattacttgtacctatcgaggacaa

SEQ ID NO: 51 - DNA, 60 bp, synthetic construct
  source 1..60: mol_type = other DNA
acagttatgttccagtgccttcataatctcatcagaattctccctataaactgtttcctc

SEQ ID NO: 52 - DNA, 60 bp, synthetic construct
  source 1..60: mol_type = other DNA
tccaggggatttaaaagagttttctgctttgagagagaaatagagagtttagaaagcaat

SEQ ID NO: 53 - DNA, 60 bp, synthetic construct
  source 1..60: mol_type = other DNA
atgtattcttggctgctatgcgtggtttttcaggaaatttaattatcttactgagatgtg

SEQ ID NO: 54 - DNA, 60 bp, synthetic construct
  source 1..60: mol_type = other DNA
gtgcagagttctatataggatgctggattagttcctttgatatttgtaaaaattccccca

SEQ ID NO: 55 - DNA, 60 bp, synthetic construct
  source 1..60: mol_type = other DNA
ggtccgtatgcggtaccttttaaaggttcagtttaatttccttcagctgtggtgaatgtt

SEQ ID NO: 56 - DNA, 60 bp, synthetic construct
  source 1..60: mol_type = other DNA
gctgtcagttttctgtggatgaagaatttcagaaactgtggaggagtgtcactgtagatt

SEQ ID NO: 57 - DNA, 60 bp, synthetic construct
  source 1..60: mol_type = other DNA
cttgcaagcatcccatggggcgggggcgggaccagggagaattaataaagttctggactt

SEQ ID NO: 58 - DNA, 60 bp, synthetic construct
  source 1..60: mol_type = other DNA
ccttaagagctacagctagagaaaccttcacaggggtggagagagcattctaaggttttt

SEQ ID NO: 59 - DNA, 60 bp, synthetic construct
  source 1..60: mol_type = other DNA
ggcaagagagaaagtgaatacgctgatataagaagcaagaaaccttccttccttccagat

SEQ ID NO: 60 - DNA, 60 bp, synthetic construct
  source 1..60: mol_type = other DNA
agaacataagagtccatatttctagtggatttgtaaaataagtgtgtgtgagacacttgc